(19) United States
(12) Patent Application Publication
(10) Pub. No.: US 2007/0032488 A1
(43) Pub. Date: Feb. 8, 2007

(54) 6-Membered aryl and heteroaryl derivatives for treating viruses

(75) Inventors: Janos Botyanszki, Fremont, CA (US); Dong-Fang Shi, Fremont, CA (US); Christopher Don Roberts, Belmont, CA (US); Franz Ulrich Schmitz, Mill Valley, CA (US)

Correspondence Address: FOLEY & LARDNER LLP, 1530 PAGE MILL ROAD, PALO ALTO, CA, 94304, US

(73) Assignee: Genelabs Technologies, Inc.

(21) Appl. No.: 11/499,461

(22) Filed: Aug. 3, 2006

Related U.S. Application Data
(60) Provisional application No. 60/705,886, filed on Aug. 5, 2005.

(51) Int. Cl. A61K 31/5375; C07D 413/02; C07D 401/02; A61K 31/454; A61K 31/4545; A61K 31/5377

(52) U.S. Cl. 514/235.2; 544/176; 514/235.5; 514/237.5; 514/326; 514/317; 544/138; 546/194; 546/207; 544/128

(57) ABSTRACT

Disclosed are compounds, compositions and methods for treating Flaviviridae family virus infections.

### CROSS-REFERENCE TO RELATED APPLICATION

This application claims the benefit under 35 U.S.C. 119(e) to co-pending provisional application U.S. Ser. No. 60/705,886 filed on Aug. 5, 2005, which is incorporated herein by reference in its entirety.

### BACKGROUND OF THE INVENTION

1. Field of the Invention

The invention relates to the field of pharmaceutical chemistry, in particular to compounds, compositions and methods for treating viral infections in mammals mediated, at least in part, by a virus in the Flaviviridae family of viruses.

### REFERENCES

The following publications are cited in this application as superscript numbers:
1. Szabo, E. et al., Pathol. Oncol. Res. 2003, 9:215-221.
2. Hoofnagle J. H., Hepatology 1997, 26:15S-20S.
3. Thomson B. J. and Finch R. G., Clin Microbial Infect. 2005, 11:86-94.
4. Moriishi K. and Matsuura Y., Antivir. Chem. Chemother. 2003, 14:285-297.
5. Fried, M. W., et al. N. Engl. J. Med 2002, 347:975-982.
6. Ni, Z. J. and Wagman, A. S. Curr. Opin. Drug Discov. Devel. 2004, 7, 446-459.
7. Beaulieu, P. L. and Tsantrizos, Y. S. Curr. Opin. Investig. Drugs 2004, 5, 838-850.
8. Griffith, R. C. et al., Ann. Rep. Med. Chem 39, 223-237, 2004.
9. Watashi, K. et al., Molecular Cell, 19, 111-122, 2005
10. Horsmans, Y. et al., Hepatology, 42, 724-731, 2005

All of the above publications are herein incorporated by reference in their entirety to the same extent as if each individual publication was specifically and individually indicated to be incorporated by reference in its entirety.

2. State of the Art

Chronic infection with HCV is a major health problem associated with liver cirrhosis, hepatocellular carcinoma and liver failure. An estimated 170 million chronic carriers worldwide are at risk of developing liver disease.^{1,2} In the United States alone 2.7 million are chronically infected with HCV, and the number of HCV-related deaths in 2000 was estimated between 8,000 and 10,000, a number that is expected to increase significantly over the next years. Infection by HCV is insidious in a high proportion of chronically infected (and infectious) carriers who may not experience clinical symptoms for many years. Liver cirrhosis can ultimately lead to liver failure. Liver failure resulting from chronic HCV infection is now recognized as a leading cause of liver transplantation.

HCV is a member of the Flaviviridae family of RNA viruses that affect animals and humans. The genome is a single ˜9.6-kilobase strand of RNA, and consists of one open reading frame that encodes for a polyprotein of ˜3000 amino acids flanked by untranslated regions at both 5′ and 3′ ends (5′- and 3′-UTR). The polyprotein serves as the precursor to at least 10 separate viral proteins critical for replication and assembly of progeny viral particles. The organization of structural and non-structural proteins in the HCV polyprotein is as follows: C-E1-E2-p7-NS2-NS3-NS4a-NS4b-NS5a-NS5b. Because the replicative cycle of HCV does not involve any DNA intermediate and the virus is not integrated into the host genome, HCV infection can theoretically be cured. While the pathology of HCV infection affects mainly the liver, the virus is found in other cell types in the body including peripheral blood lymphocytes.^{3,4}

At present, the standard treatment for chronic HCV is interferon alpha (IFN-alpha) in combination with ribavirin and this requires at least six (6) months of treatment. IFN-alpha belongs to a family of naturally occurring small proteins with characteristic biological effects such as antiviral, immunoregulatory and antitumoral activities that are produced and secreted by most animal nucleated cells in response to several diseases, in particular viral infections. IFN-alpha is an important regulator of growth and differentiation affecting cellular communication and immunological control. Treatment of HCV with interferon has frequently been associated with adverse side effects such as fatigue, fever, chills, headache, myalgias, arthralgias, mild alopecia, psychiatric effects and associated disorders, autoimmune phenomena and associated disorders and thyroid dysfunction. Ribavirin, an inhibitor of inosine 5′-monophosphate dehydrogenase (IMPDH), enhances the efficacy of IFN-alpha in the treatment of HCV. Despite the introduction of ribavirin, more than 50% of the patients do not eliminate the virus with the current standard therapy of interferon-alpha (IFN) and ribavirin. By now, standard therapy of chronic hepatitis C has been changed to the combination of pegylated IFN-alpha plus ribavirin. However, a number of patients still have significant side effects, primarily related to ribavirin. Ribavirin causes significant hemolysis in 10-20% of patients treated at currently recommended doses, and the drug is both teratogenic and embryotoxic. Even with recent improvements, a substantial fraction of patients do not respond with a sustained reduction in viral load⁵ and there is a clear need for more effective antiviral therapy of HCV infection.

A number of approaches are being pursued to combat the virus. They include, for example, application of antisense oligonucleotides or ribozymes for inhibiting HCV replication. Furthermore, low-molecular weight compounds that directly inhibit HCV proteins and interfere with viral replication are considered as attractive strategies to control HCV infection. Among the viral targets, the NS3/4a protease/helicase and the NS5b RNA-dependent RNA polymerase are considered the most promising viral targets for new drugs.⁶⁻⁸

Besides targeting viral genes and their transcription and translation products, antiviral activity can also be achieved by targeting host cell proteins that are necessary for viral replication. For example, Watashi et al.⁹ show how antiviral activity can be achieved by inhibiting host cell cyclophilins. Alternatively, a potent TLR7 agonist has been shown to reduce HCV plasma levels in humans.¹⁰

However, none of the compounds described above have progressed beyond clinical trials.^{6,8}

In view of the worldwide epidemic level of HCV and other members of the Flaviviridae family of viruses, and further in view of the limited treatment options, there is a strong need for new effective drugs for treating infections cause by these viruses.

### SUMMARY OF THE INVENTION

The present invention is directed to novel compounds, compositions, and methods for treating viral infections in mammals mediated, at least in part, by a member of the Flaviviridae family viruses such as HCV. Specifically, compounds of this invention are represented by formula I, II, or III: 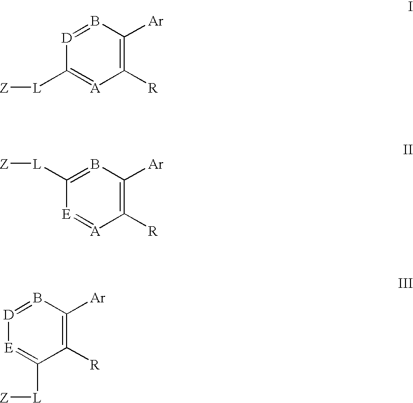
wherein:

B and D are independently N or C-L¹-R¹;

with the proviso that at least one of B or D is N or CH;

A and E are independently N or C—R²;

R is selected from the group consisting of alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl;

R¹ is selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, —COOH, —COOR^{1a}, —CONR³R⁴ and —NR³R⁴; where each of R^{1a}, R³, and R⁴ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or, alternatively, R³ and R⁴ may optionally be joined together with the nitrogen atom bound thereto to form a heterocyclic, substituted heterocyclic, heteroaryl or substituted heteroaryl group;

R² is selected from the group consisting of hydrogen, halo, C₁-C₂ alkyl, substituted C₁-C₂ alkyl, C₂-C₃ alkenyl, substituted C₂-C₃ alkenyl, cyclopropyl, and substituted cyclopropyl;

L and L¹ are independently selected from the group consisting of a bond, C₁-C₃ alkylene, substituted C₁-C₃ alkylene, C₂-C₃ alkenylene, substituted C₂-C₃ alkenylene, C₂-C₃ alkynylene, substituted C₂-C₃ alkynylene, C₃-C₆ cycloalkylene, substituted C₃-C₆ cycloalkylene, C₄-C₆ cycloalkenylene, C₄-C₆ substituted cycloalkenylene, arylene, substituted arylene, heteroarylene, and substituted heteroarylene;

Z is selected from the group consisting of:

(a) hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;

(b) COOH and COOR^{z}, wherein R^{z} is selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl;

(c) —C(X¹)NR⁵R⁶, wherein X¹ is ═O, ═NH, or ═N-alkyl, R⁵ and R⁶ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁵ and R⁶ together with the nitrogen atom pendent thereto, form a heterocyclic, a substituted heterocyclic, a heteroaryl or a substituted heteroaryl ring group;

(d) —C(X²)NR⁷S(O)₂R⁸, wherein X² is selected from ═O, ═NR⁹, and ═S, wherein R⁹ is hydrogen, alkyl, or substituted alkyl; R⁸ is selected from alkyl, substituted alkyl, aryl, substituted aryl, heteroaryl, substituted heteroaryl, heterocyclic, substituted heterocyclic, and NR¹⁰R¹¹ wherein each of R⁷, R¹⁰, and R¹¹ is independently hydrogen, alkyl, substituted alkyl, cycloalkyl, or substituted cycloalkyl, and wherein each of R⁷ and R¹⁰ is optionally substituted with one to three substituents selected from the group consisting of halo, hydroxyl, carboxy, carboxy ester, alkyl, alkoxy, amino, and substituted amino; or alternatively, R⁷ and R¹⁰ or R¹⁰ and R¹¹ together with the atoms bound thereto join together to form an optionally substituted heterocyclic group;

(e) —C(X³)—N(R¹²)CR¹³R^{13′}C(═O)R¹⁴, wherein X³ is selected from ═O, ═S, and ═NR¹⁵, where R¹⁵ is hydrogen or alkyl, R¹⁴ is selected from —OR¹⁶ and —NR¹⁰R¹¹ where R¹⁶ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁰ and R¹¹ are as defined above; R¹³ and R^{13′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or,

alternatively, R¹³ and R^{13′} as defined are taken together with the carbon atom pendent thereto to form a cycloalkyl, substituted cycloalkyl, heterocyclic or substituted heterocyclic group; or, still further alternatively, one of R¹³ or R^{13′} is hydrogen, alkyl or substituted alkyl, and the other is joined, together with the carbon atom pendent thereto, with either the R¹⁶ and the oxygen atom pendent thereto or R¹⁰ and the nitrogen atom pendent thereto to form a heterocyclic or substituted heterocyclic group;
R¹² is selected from hydrogen and alkyl or, when R¹³ and R¹³ are not taken together to form a ring and when R¹³ or R^{13′} and R¹⁰ or R¹¹ are not joined to form a heterocyclic or substituted heterocyclic group, then R², together with the nitrogen atom pendent thereto, may be taken together with one of R¹³ and R^{13′} to form a heterocyclic or substituted heterocyclic ring group;

(f) —C(X²)—N(R²)CR¹⁷R¹⁸R¹⁹, wherein X² and R¹² are defined above, and R¹⁷, R¹⁸ and R¹⁹ are independently alkyl, substituted alky, aryl, substituted aryl, heterocyclic, substituted heterocyclic, heteroaryl and substituted heteroaryl, or R¹⁷ and R¹⁸ together with the carbon atom pendent thereto form a cycloalkyl, substituted cycloalkyl, heterocyclic or substituted heterocyclic group; and

(g) carboxylic acid isostere;

with the proviso that when L is a bond, Z is not hydrogen; and

Ar is selected from the group consisting of aryl, heteroaryl, substituted aryl, and substituted heteroaryl;

or a pharmaceutically acceptable salt, ester, stereoisomer, prodrug, or tautomer thereof.

### DETAILED DESCRIPTION OF THE INVENTION

The invention is directed to compounds, compositions and methods for treating Flaviviridae family viral infections.

In one embodiment, the present invention provides compounds represented by formula I, II, or III: 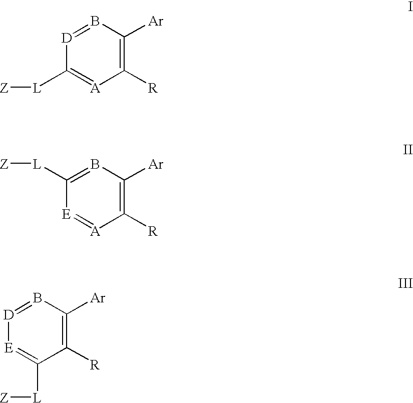
wherein:

B and D are independently N or C-L¹-R¹;

with the proviso that at least one of B or D is N or CH;

A and E are independently N or C—R²;

R is selected from the group consisting of alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl;

R¹ is selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, —COOH, —COOR^{1a}, —CONR³R⁴ and —NR³R⁴; where each of R^{1a}, R³, and R⁴ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or, alternatively, R³ and R⁴ may optionally be joined together with the nitrogen atom bound thereto to form a heterocyclic, substituted heterocyclic, heteroaryl or substituted heteroaryl group;

R² is selected from the group consisting of hydrogen, halo, C₁-C₂ alkyl, substituted C₁-C₂ alkyl, C₂-C₃ alkenyl, substituted C₂-C₃ alkenyl, cyclopropyl, and substituted cyclopropyl;

L and L¹ are independently selected from the group consisting of a bond, C₁-C₃ alkylene, substituted C₁-C₃ alkylene, C₂-C₃ alkenylene, substituted C₂-C₃ alkenylene, C₂-C₃ alkynylene, substituted C₂-C₃ alkynylene, C₃-C₆ cycloalkylene, substituted C₃-C₆ cycloalkylene, C₄-C₆ cycloalkenylene, C₄-C₆ substituted cycloalkenylene, arylene, substituted arylene, heteroarylene, and substituted heteroarylene;

Z is selected from the group consisting of:

(a) hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;

(b) COOH and COOR^{z}, wherein R^{z} is selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl;

(c) —C(X¹)NR⁵R⁶, wherein X¹ is ═O, ═NH, or ═N-alkyl, R⁵ and R⁶ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁵ and R⁶ together with the nitrogen atom pendent thereto, form a heterocyclic, a substituted heterocyclic, a heteroaryl or a substituted heteroaryl ring group;

(d) —C(X²)NR⁷S(O)₂R⁸, wherein X² is selected from ═O, ═NR⁹, and ═S, wherein R⁹ is hydrogen, alkyl, or substituted alkyl; R⁸ is selected from alkyl, substituted alkyl, aryl, substituted aryl, heteroaryl, substituted heteroaryl, heterocyclic, substituted heterocyclic, and NR¹⁰R¹¹ wherein each of R⁷, R¹⁰, and R¹¹ is independently hydrogen, alkyl, substituted alkyl, cycloalkyl, or substituted cycloalkyl, and wherein each of R⁷ and R¹⁰ is optionally substituted with one to three substituents selected from the group consisting of halo, hydroxyl, carboxy, carboxy ester, alkyl, alkoxy, amino, and substituted amino; or alternatively, R⁷ and R¹⁰ or R¹⁰ and R¹ together with the atoms bound thereto join together to form an optionally substituted heterocyclic group;

(e) —C(X³)—N(R¹²)CR¹³R^{13′}C(═O)R¹⁴, wherein X³ is selected from ═O, ═S, and ═NR¹⁵, where R¹⁵ is hydrogen or alkyl, R¹⁴ is selected from —OR¹⁶ and —NR¹⁰R¹¹ where R¹⁶ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁰ and R¹¹ are as defined above;

R¹³ and R^{13′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or,

alternatively, R¹³ and R^{13′} as defined are taken together with the carbon atom pendent thereto to form a cycloalkyl, substituted cycloalkyl, heterocyclic or substituted heterocyclic group; or, still further alternatively, one of R¹³ or R^{13′} is hydrogen, alkyl or substituted alkyl, and the other is joined, together with the carbon atom pendent thereto, with either the R¹⁶ and the oxygen atom pendent thereto or R¹⁰ and the nitrogen atom pendent thereto to form a heterocyclic or substituted heterocyclic group;

R¹² is selected from hydrogen and alkyl or, when R¹³ and R^{13′} are not taken together to form a ring and when R¹³ or R^{13′} and R¹⁰ or R¹¹ are not joined to form a heterocyclic or substituted heterocyclic group, then R¹², together with the nitrogen atom pendent thereto, may be taken together with one of R¹³ and R^{13′} to form a heterocyclic or substituted heterocyclic ring group;

(f) —C(X²)—N(R¹²)CR¹⁷R¹⁸R¹⁹, wherein X² and R¹² are defined above, and R¹⁷, R¹⁸ and R¹⁹ are independently alkyl, substituted alky, aryl, substituted aryl, heterocyclic, substituted heterocyclic, heteroaryl and substituted heteroaryl, or R¹⁷ and R¹⁸ together with the carbon atom pendent thereto form a cycloalkyl, substituted cycloalkyl, heterocyclic or substituted heterocyclic group; and

(g) carboxylic acid isostere;

with the proviso that when L is a bond, Z is not hydrogen; and

Ar is selected from the group consisting of aryl, heteroaryl, substituted aryl, and substituted heteroaryl;

or a pharmaceutically acceptable salt, ester, stereoisomer, prodrug, or tautomer thereof.

In some embodiments, the invention provides compounds wherein one of B and D is C-L¹-R¹ and the other of B and D is CH.

In other embodiments, the invention provides compounds wherein A and E are C—R². In still other embodiments A and E are CH. In some aspects, R² is hydrogen, L¹ is a bond, —CH₂—, —CH₂CH₂—, cis or trans —CH═CH—, cis or trans —(CH₃)C═CH—, cis or trans —CH═C(CH₃)—, or —CC—, and R¹ is hydrogen or —CONR³R⁴. In other aspects R³ and R⁴ together with the nitrogen atom bound thereto form a substituted or unsubstituted heterocyclic group. In still other aspects, R³ is alkyl and R⁴ is (heterocyclic)alkyl or (substituted heterocyclic)alkyl.

In some embodiments, Z is —COOH, —COOR^{z}, 1H-tetrazol-5-yl, —C(O)NHSO₂CF₃,

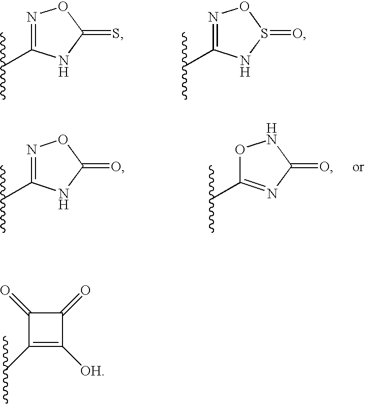

In some aspects, Z is —COOH.

In other aspects, L is a bond, —CH₂—, cis or trans —CH═CH—, cis or trans —(CH₃)C═CH—, or cis or trans —CH═C(CH₃)—.

In some embodiments, R is substituted or unsubsituted cylcoalkyl, substituted or unsubsituted heterocyclic, or substituted and unsubstituted cyclohexyl. In some aspects, R is cyclohexyl.

In some embodiments, Ar is a substituted or unsubstituted five or six membered aryl or heteroaryl group, or a substituted or unsubstituted bicyclic [6,6], [5,6], or [6,5] aryl or heteroaryl group.

The present invention also provides compounds, pharmaceutically acceptable salts, esters, stereoisomers, prodrugs, or tautomers thereof having formula IV, V, or VI:

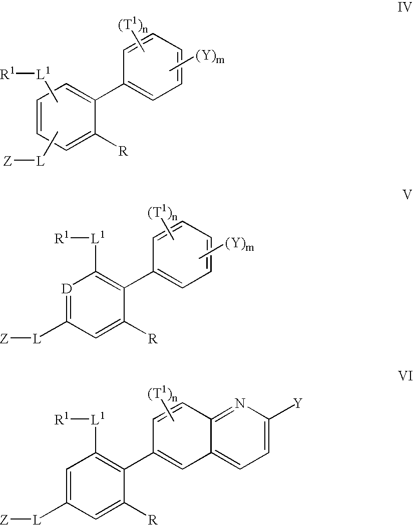

wherein D, Z, L, L¹, R, and R¹ are previously defined; each T¹ is independently selected from the group consisting of alkyl, substituted alkyl, alkoxy, substituted alkoxy, amino, substituted amino, cyano, carboxy, carboxy ester, halo, hydroxyl, heterocyclic, substituted hetereocyclic, and nitro; Y is selected from the group consisting of aryl, heteroaryl, substituted aryl, and substituted heteroaryl; n is an integer equal to 0, 1, or 2, and m is an integer equal to 0 or 1.

In some embodiments, L¹ is a bond, cyclopropyl, —CH₂—, —CH₂CH₂—, cis or trans —CH═CH—, cis or trans —(CH₃)C═CH—, cis or trans —CH═C(CH₃)—, or —CC— and R¹ is hydrogen or —CONR³R⁴. In other embodiments, R³ and R⁴ together with the nitrogen atom bound thereto form a substituted or unsubstituted heterocyclic group. In still other embodiments R³ is alkyl and R⁴ is alkyl substituted with a heterocyclic or substituted heterocyclic group.

In some embodiments, the invention provides compounds where Z is a carboxylic acid isostere. In other embodiments, Z is —COOH, —COOR^{z} (where R^{z} is as defined above), 1H-tetrazol-5-yl, —C(O)NHSO₂CF₃,

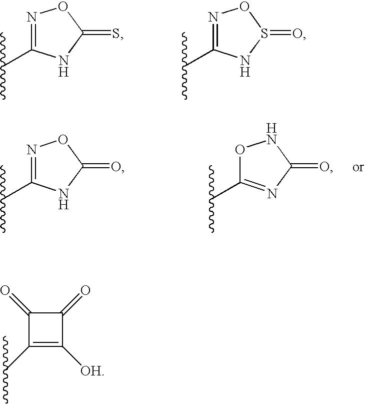

In some embodiments, the invention provides compounds where L is a bond, —CH₂—, cis or trans —CH═CH—, cis or trans —(CH₃)C═CH—, or cis or trans —CH═C(CH₃)—.

In other embodiments, the invention provides compounds where R is substituted or unsubsituted cylcoalkyl, substituted or unsubsituted heterocyclic, or substituted and unsubstituted cyclohexyl. In some embodiments R is cyclohexyl.

In some embodiments, the invention provides compounds where Ar is a substituted or unsubstituted five or six membered aryl or heteroaryl group, or a substituted or unsubstituted bicyclic [6,6], [5,6], or [6,5] aryl or heteroaryl group.

In still other embodiments, the invention provides compounds where Ar has the formula (H1) 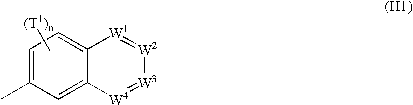
where each of W¹, W², W³ and W⁴ are independently selected from N,N-oxide, CH, CT², and C—Y, provided that no more than 2 of W¹, W², W³ and W⁴ are N; provided that at least one of W¹, W², W³ and W⁴ is C—Y; and further provided wherein no more than one N in (H1) is N-oxide; each of T¹ and T² are independently selected from the group consisting of alkyl, substituted alkyl, alkoxy, substituted alkoxy, amino, substituted amino, cyano, carboxy, carboxy ester, halo, hydroxyl, heterocyclic, substituted hetereocyclic, and nitro; Y is selected from the group consisting of aryl, heteroaryl, substituted aryl, and substituted heteroaryl; and n is an integer equal to 0, 1, or 2.

In some embodiments, Ar has the formula (H2) 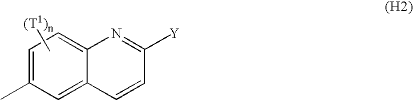
where T¹, n, and Y are defined as for formula (H1). In still other embodiments, n is 0.

In some embodiments, the invention provides compounds where Y is selected from the group consisting of 3-(2-methoxyethoxy)-5-(4′-chlorophenyl)phenyl, 3-methoxy-5-(4′-chlorophenyl)phenyl, and 2,4-dimethylthiazol-5-yl.

The present invention further provides compounds and their pharmaceutically acceptable salt, ester, stereoisomer, prodrug, or tautomer thereof resulting from a combination of any of the variables relating to the atoms and substituents of formulas I-VI, particularly those variables in the specified embodiments above. Compounds of this invention having such combinations include, by way of example, compounds where:

Z is a carboxylic isostere, R is cycloalkyl, and Ar is substituted or unsubstituted phenyl or substituted or unsubstituted quinolin-6-yl;

Z is COOH, R is cyclohexyl, and Ar is substituted or unsubstituted phenyl or substituted or unsubstituted quinolin-6-yl;

Z is COOH, R is cyclohexyl, Ar is substituted or unsubstituted phenyl or substituted or unsubstituted quinolin-6-yl, and L is a bond, —CH₂—, cis or trans —CH═CH—, cis or trans —(CH₃)C═CH—, or cis or trans —CH═C(CH₃)—;

Z is COOH, R is cyclohexyl, Ar is substituted or unsubstituted phenyl or substituted or unsubstituted quinolin-6-yl, L is a bond, —CH₂—, cis or trans —CH═CH—, cis or trans —(CH₃)C═CH—, or cis or trans —CH═C(CH₃)—, L¹ is a bond, —CH₂—, —CH₂CH₂—, cis or trans —CH═CH—, cis or trans —(CH₃)C═CH—, cis or trans —CH═C(CH₃)—, or —CC—, and R¹ is hydrogen or —CONR³R⁴ where R³ and R⁴ are as defined above;

Z is COOH, R is cyclohexyl, Ar is substituted or unsubstituted phenyl or substituted or unsubstituted quinolin-6-yl, L is a bond, —CH₂—, cis or trans —CH═CH—, cis or trans —(CH₃)C═CH—, or cis or trans —CH═C(CH₃)—, L¹ is a bond, —CH₂—, —CH₂CH₂—, cis or trans —CH═CH—, cis or trans —(CH₃)C═CH—, cis or trans —CH═C(CH₃)—, or —CC—, R¹ is —CONR³R⁴, and R³ and R⁴ together with the nitrogen to which they are attached form a substituted or unsubsituted heterocyclic ring;

Z is COOH, R is cyclohexyl, Ar is substituted or unsubstituted phenyl or substituted or unsubstituted quinolin-6-yl, L is a bond, L¹ is a bond, —CH₂—, —CH₂CH₂—, cis or trans —CH═CH—, cis or trans —(CH₃)C═CH—, cis or trans —CH═C(CH₃)—, or —CC—, R¹ is —CONR³R⁴, R³ is alkyl, and R⁴ is (heterocyclic)alkyl or (substituted heterocyclic)alkyl;

Z is COOH, R is cyclohexyl, Ar is substituted or unsubstituted phenyl, L is a bond or —CH₂—, cis or trans —CH═CH—, cis or trans —(CH₃)C═CH—, or cis or trans —CH═C(CH₃)—, L¹ is a bond, —CH₂—, —CH₂CH₂—, cis or trans —CH═CH—, cis or trans —(CH₃)C═CH—, cis or trans —CH═C(CH₃)—, or —CC—, R¹ is —CONR³R⁴, R³ is alkyl, and R⁴ is (heterocyclic)alkyl or (substituted heterocyclic)alkyl.

The present invention provides compounds or their pharmaceutically acceptable salt, ester, stereoisomer, prodrug, or tautomer thereof of formula V having the following structure:

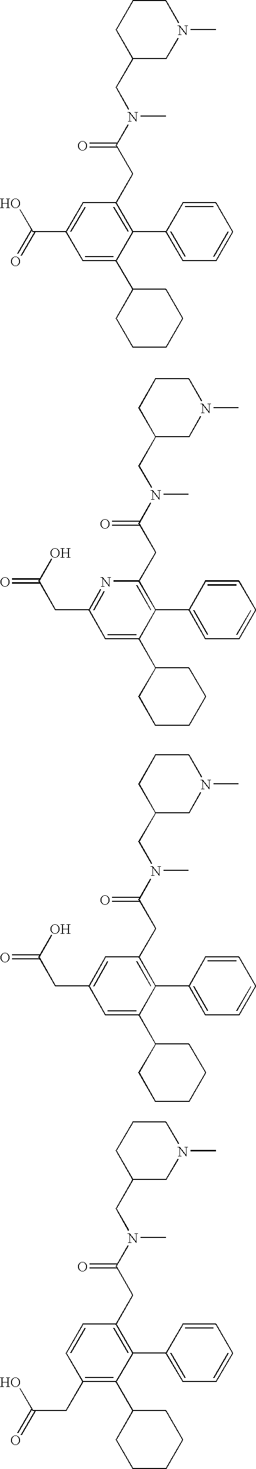

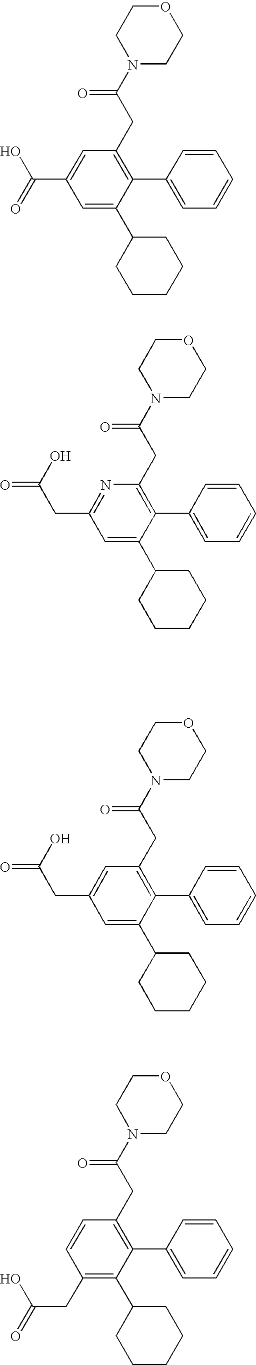

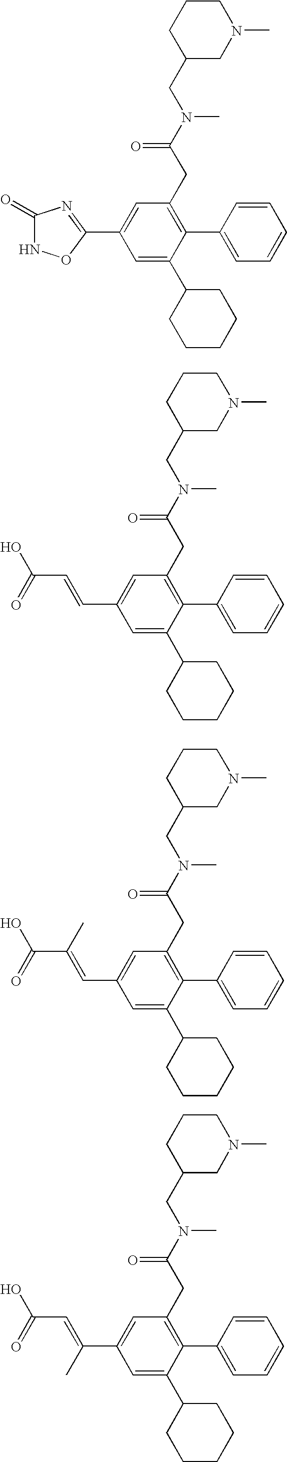

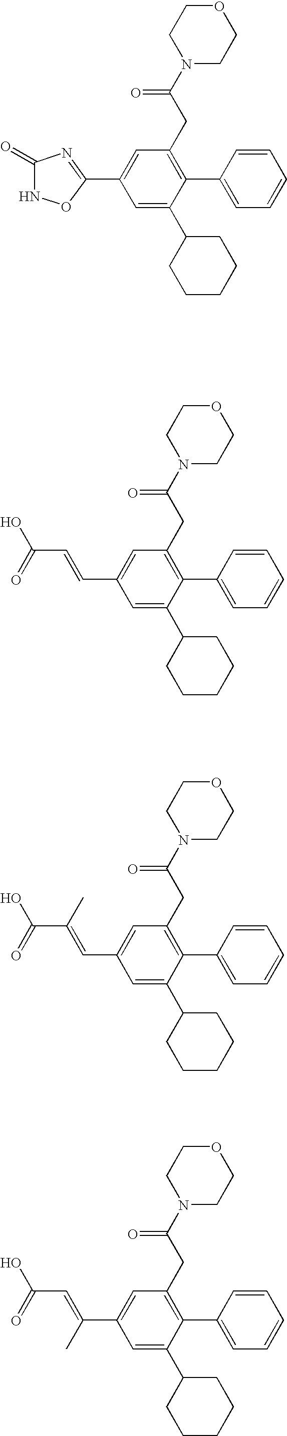

Other compounds of the invention or their pharmaceutically acceptable salt, ester, stereoisomer, prodrug, or tautomer thereof are compounds of the formula VI set forth in Table I and II below.

TABLE I

| Compd | | |
|---|---|---|
| # | Structure | Name |
| 1.8b | 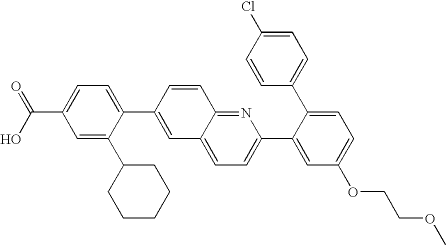 | 3-cyclohexyl-4-(2-(3-(2- methoxyethoxy)-6-(4′- chlorophenyl)phenyl)quinolin- 6-yl)benzoic acid |
| 1.8a | 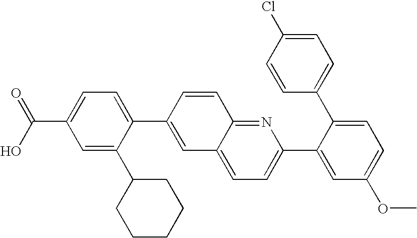 | 3-cyclohexyl-4-(2-(3- methoxy-6-(4′- chlorophenyl)phenyl)quinolin- 6-yl)benzoic acid |
| 2.9b | 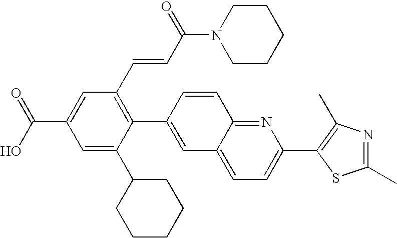 | 3-cyclohexyl-4-(2-(2,4- dimethylthiazol-5-yl)quinolin- 6-yl)-5-((E)-3-oxo-3- (piperidin-1-yl)prop-1- enyl)benzoic acid |
| 2.9a | 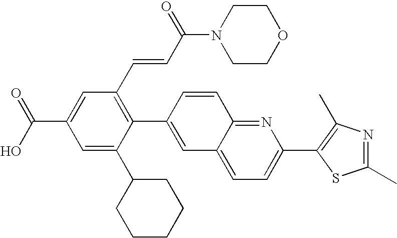 | 3-cyclohexyl-4-(2-(2,4- dimethylthiazol-5-yl)quinolin- 6-yl)-5-((E)-3-morpholino-3- oxoprop-1-enyl)benzoic acid |
| 4.4b | 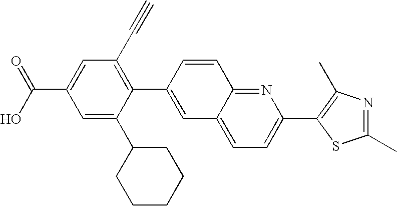 | 3-cyclohexyl-5-ethynyl-4-(2- (2,4-dimethylthiazol-5- yl)quinolin-6-yl)benzoic acid |
| 4.6b | 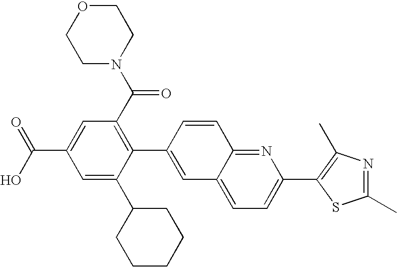 | 3-cyclohexyl-4-(2-(2,4- dimethylthiazol-5-yl)-5-(1- morpholino-1-oxomethyl)- quinolin-6-yl)benzoic acid |
| 7 | 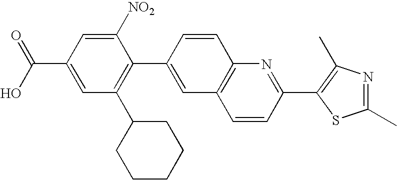 | 3-cyclohexyl-4-[2-(2,4- dimethyl-thiazol-5-yl)- quinolin-6-yl]-5-nitro-benzoic acid |
| 8 | 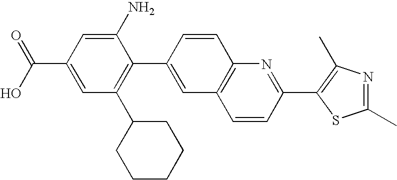 | 3-amino-5-cyclohexyl-4-[2- (2,4-dimethyl-thiazol-5-yl)- quinolin-6-yl]-benzoic acid |
| 9 | 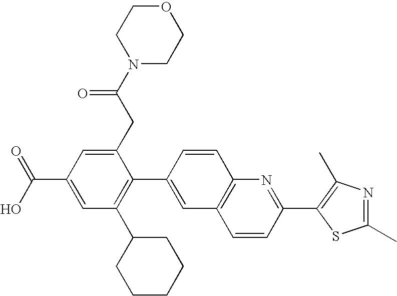 | 3-cyclohexyl-4-[2-(2,4- dimethyl-thiazol-5-yl)- quinolin-6-yl]-5-(2- morpholin-4-yl-2-oxo-ethyl)- benzoic acid |
| 10 | 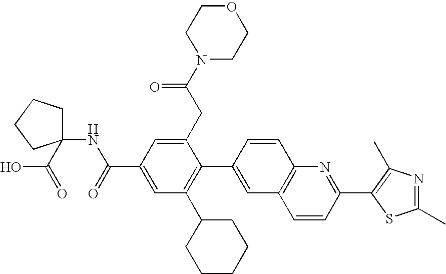 | 1-[3-cyclohexyl-4-[2-(2,4- dimethyl-thiazol-5-yl)- quinolin-6-yl]-5-(2- m\orpholin-4-yl-2-oxo-ethyl)- benzoylamino]- cyclopentanecarboxylic acid |
| 11 | 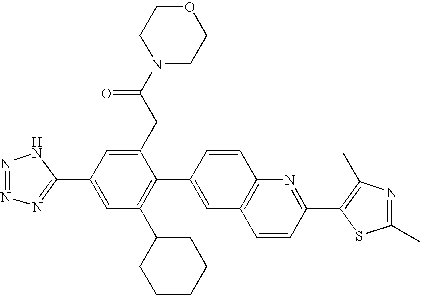 | 2-[3-cyclohexyl-2-[2-(2,4- dimethyl-thiazol-5-yl)- quinolin-6-yl]-5-(1H-tetrazol 5-yl)-phenyl]-1-morpholin-4- yl-ethanone |

TABLE 2

| Compd | | |
|---|---|---|
| # | Structure | Name |
| 12 | 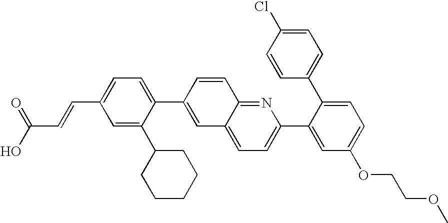 | 3-(4-{2-[4′-chloro-4-(2- methoxy-ethoxy)-biphenyl- 2-yl]-quinolin-6-yl}-3- cyclohexyl-phenyl)-acrylic acid |
| 13 | 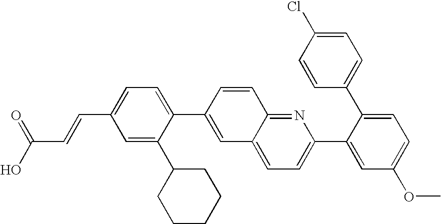 | 3-{4-[2-(4′-chloro-4- methoxy-biphenyl-2-yl)- quinolin-6-yl]-3- cyclohexyl-phenyl}-acrylic acid |
| 14 | 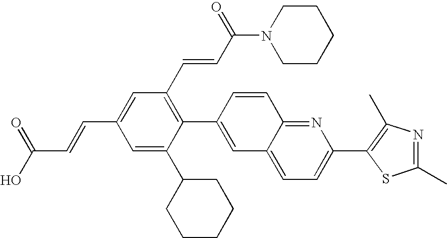 | 3-[3-cyclohexyl-4-[2-(2,4- dimethyl-thiazol-5-yl)- quinolin-6-yl]-5-(3-oxo-3- piperidin-1-yl-propenyl)- phenyl]-acrylic acid |
| 15 | 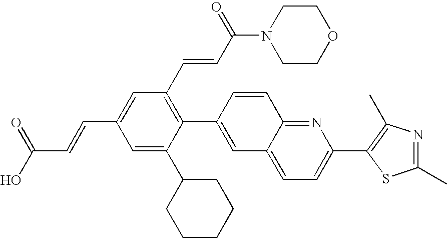 | 3-[3-cyclohexyl-4-[2-(2,4- dimethyl-thiazol-5-yl)- quinolin-6-yl]-5-(3- morpholin-4-yl-3-oxo propenyl)-phenyl]-acrylic acid |
| 16 | 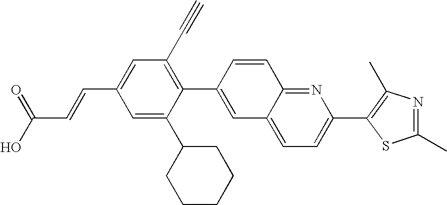 | 3-{3-cyclohexyl-4-[2-(2,4- dimethyl-thiazol-5-yl)- quinolin-6-yl]-5-ethynyl- phenyl}-acrylic acid |
| 17 | 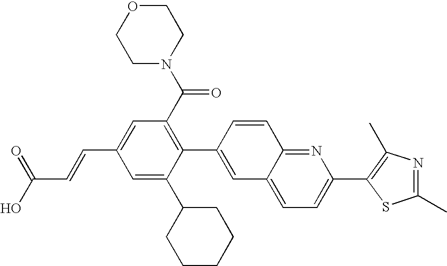 | 3-[3-cyclohexyl-4-[2-(2,4- dimethyl-thiazol-5-yl)- quinolin-6-yl]-5- (morpholine-4-carbonyl)- phenyl]-acrylic acid |
| 18 | 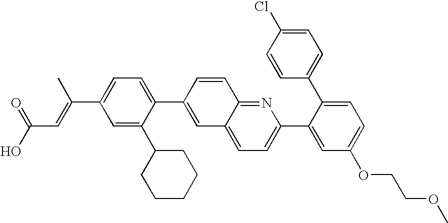 | 3-(4-{2-[4′-chloro-4-(2- methoxy-ethoxy)-biphenyl- 2-yl]-quinolin-6-yl}-3- cyclohexyl-phenyl)-but-2- enoic acid |
| 19 | 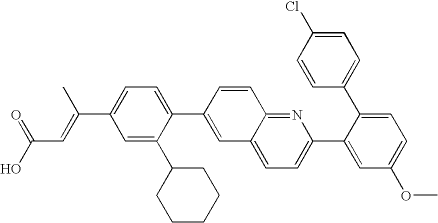 | 3-{4-[2-(4′-chloro-4- methoxy-biphenyl-2-yl)- quinolin-6-yl]-3- cyclohexyl-phenyl}-but-2- enoic acid |
| 20 | 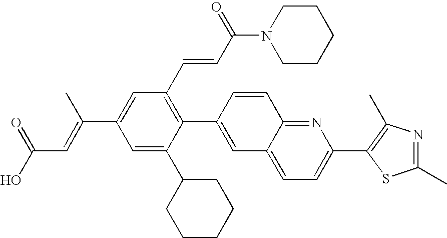 | 3-[3-cyclohexyl-4-[2-(2,4- dimethyl-thiazol-5-yl)- quinolin-6-yl]-5-(3-oxo-3- piperidin-1-yl-propenyl)- phenyl]-but-2-enoic acid |
| 21 | 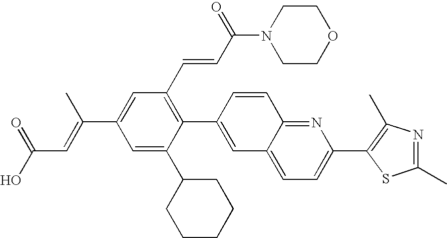 | 3-[3-cyclohexyl-4-[2-(2,4- dimethyl-thiazol-5-yl)- quinolin-6-yl]-5-(3- morpholin-4-yl-3-oxo- propenyl)-phenyl]-but-2- enoic acid |
| 22 | 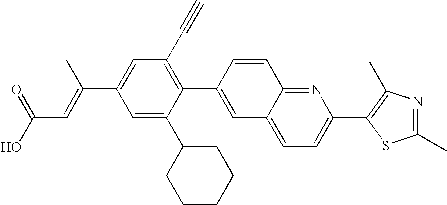 | 3-{3-cyclohexyl-4-[2-(2,4- dimethyl-thiazol-5-yl)- quinolin-6-yl]-5-ethynyl- phenyl}-but-2-enoic acid |
| 23 | 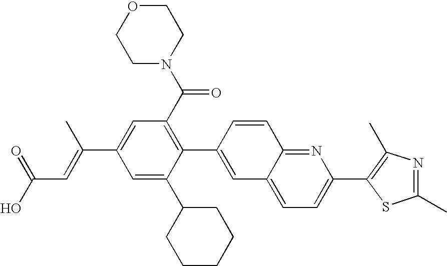 | 3-[3-cyclohexyl-4-[2-(2,4- dimethyl-thiazol-5-yl)- quinolin-6-yl]-5- (morpholine-4-carbonyl)- phenyl]-but-2-enoic acid |

Also provided are alkynyl compounds corresponding to the compounds in Table II wherein the alkenylene group L is replaced with an alkynylene group.

This invention is also directed to pharmaceutical compositions comprising a pharmaceutically acceptable diluent and a therapeutically effective amount of one of the compounds described herein or mixtures of one or more of such compounds.

This invention is further directed to uses of the compounds as described herein or mixtures of one or more of such compounds in the preparation of a medicament for treating a viral infection mediated, at least in part, by a virus in the Flaviviridae family of viruses, such as HCV.

This invention is still further directed to methods for treating a viral infection mediated at least in part by a virus in the Flaviviridae family of viruses, such as HCV, in mammals which methods comprise administering to a mammal, that has been diagnosed with said viral infection or is at risk of developing said viral infection, a pharmaceutical composition comprising a pharmaceutically acceptable diluent and a therapeutically effective amount of one of the compounds described herein or mixtures of one or more of such compounds.

In yet another embodiment of the invention, methods of treating or preventing viral infections in mammals are provided wherein the compounds of this invention are administered in combination with the administration of a therapeutically effective amount of one or more agents active against HCV. Active agents against HCV include ribavirin, levovirin, viramidine, thymosin alpha-1, an inhibitor of NS3 serine protease, and inhibitor of inosine monophosphate dehydrogenase, interferon-alpha, pegylated interferon-alpha, alone or in combination with ribavirin or viramidine. Preferably, the additional agent active against HCV is interferon-alpha or pegylated interferon-alpha alone or in combination with ribavirin or viramidine.

Definitions

Unless otherwise indicated, this invention is not limited to any particular composition or pharmaceutical carrier, as such may vary. It is also to be understood that the terminology used herein is for the purpose of describing particular embodiments only and is not intended to limit the scope of the present invention.

It must be noted that as used herein and in the claims, the singular forms “a,” “and” and “the” include plural referents unless the context clearly dictates otherwise. Thus, for example, reference to “pharmaceutically acceptable diluent” in a composition includes two or more pharmaceutically acceptable diluents, and so forth.

In this specification and in the claims that follow, reference will be made to a number of terms that shall be defined to have the following meanings:

As used herein, “alkyl” refers to monovalent hydrocarbyl groups having from 1 to 10 carbon atoms, preferably from 1 to 5 carbon atoms, more preferably 1 to 3 carbon atoms, and also more preferably from 1 to 2 carbon atoms. This term is exemplified by groups such as methyl, ethyl, n-propyl, iso-propyl, n-butyl, t-butyl, n-pentyl and the like.

“Substituted alkyl” refers to an alkyl group having from 1 to 3, and preferably 1 to 2, substituents selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxy, carboxy ester, cycloalkyl, substituted cycloalkyl, heteroaryl, substituted heteroaryl, heterocyclic, and substituted heterocyclic.

“Alkylene” and “substituted alkylene” refer to divalent alkyl and substituted alkyl groups as defined above. Preferred alkylene and substituted alkylene groups have one to three or two to three carbon atoms.

“Alkoxy” refers to the group “alkyl-O-” which includes, by way of example, methoxy, ethoxy, n-propoxy, iso-propoxy, n-butoxy, t-butoxy, sec-butoxy, n-pentoxy and the like.

“Substituted alkoxy” refers to the group “substituted alkyl-O-”.

“Acyl” refers to the groups H—C(O)—, alkyl-C(O)—, substituted alkyl-C(O)—, alkenyl-C(O)—, substituted alkenyl-C(O)—, alkynyl-C(O)—, substituted alkynyl-C(O)—, cycloalkyl-C(O)—, substituted cycloalkyl-C(O)—, aryl-C(O)—, substituted aryl-C(O)—, heteroaryl-C(O)—, substituted heteroaryl-C(O), heterocyclic-C(O)—, and substituted heterocyclic-C(O)—.

“Acylamino” refers to the group —C(O)NR^{f′}R^{g′} where R^{f′} and R^{9′} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{f′} and R^{g′} are joined to form together with the nitrogen atom a heterocyclic or substituted heterocyclic ring.

“Acyloxy” refers to the groups alkyl-C(O)O—, substituted alkyl-C(O)O—, alkenyl-C(O)O—, substituted alkenyl-C(O)O—, alkynyl-C(O)O—, substituted alkynyl-C(O)O—, aryl-C(O)O—, substituted aryl-C(O)O—, cycloalkyl-C(O)O—, substituted cycloalkyl-C(O)O—, heteroaryl-C(O)O—, substituted heteroaryl-C(O)O—, heterocyclic-C(O)O—, and substituted heterocyclic-C(O)O—.

“Alkenyl” refers to hydrocarbyl groups having from 2 to 10 carbon atoms, preferably having from 2 to 6 carbon atoms, and more preferably 2 to 4 carbon atoms and having at least 1 and preferably from 1-2 sites of alkenyl unsaturation wherein each site of unsaturation independently has either cis or trans orientation or a mixture thereof.

“Substituted alkenyl” refers to alkenyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxy, carboxy ester, cycloalkyl, substituted cycloalkyl, heteroaryl, substituted heteroaryl, heterocyclic, and substituted heterocyclic provided that any hydroxyl substitution is not pendent to a vinyl carbon atom.

“Alkenylene” and “substituted alkenylene” refer to divalent alkenyl and substituted alkenyl groups as defined above. Preferred alkenylene and substituted alkenylene groups have two to three carbon atoms.

“Alkenyloxy” refers to the group alkenyl-O—.

“Alkylaryloxy” refers to the group alkyl-arylene-O—.

“Alkylthio” refers to the group alkyl-S—.

“Arylalkyloxy” refers to the group aryl-alkylene-O—

“Alkynyl” refers to hydrocarbyl groups having from 2 to 10 carbon atoms, preferably having from 2 to 6 carbon atoms, and more preferably 2 to 3 carbon atoms and having at least 1 and preferably from 1-2 sites of alkynyl unsaturation.

“Substituted alkynyl” refers to alkynyl groups having from 1 to 3 substituents, and preferably 1 to 2 substituents, selected from the group consisting of alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxy, carboxy ester, cycloalkyl, substituted cycloalkyl, heteroaryl, substituted heteroaryl, heterocyclic, and substituted heterocyclic provided that any hydroxyl substitution is not pendent to an acetylenic carbon atom.

“Alkynylene” and “substituted alkynylene” refer to divalent alkynyl and substituted alkynyl groups as defined above. Preferred alkynlene and substituted alkynylene groups have two to three carbon atoms.

“Amino” refers to the group —NH₂.

“Substituted amino” refers to the group —NR^{h′}R^{i′} where R^{h′} and R^{i′} are independently selected from the group consisting of hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, substituted heterocyclic and where R^{h′} and R^{i′} are joined, together with the nitrogen bound thereto to form a heterocyclic or substituted heterocyclic group provided that R^{h′} and R^{i′} are both not hydrogen. When R^{h′} is hydrogen and R^{i′} is alkyl, the substituted amino group is sometimes referred to herein as alkylamino. When R^{h′} and R^{i′} are alkyl, the substituted amino group is sometimes referred to herein as dialkylamino.

“Aminoacyl” refers to the groups —NR^{j′}C(O)alkyl, —NR^{j′}C(O)substituted alkyl, —NR^{j′}C(O)-cycloalkyl, —NR^{j′}C(O)substituted cycloalkyl, —NR^{j′}C(O)alkenyl, —NR^{j′}C(O)substituted alkenyl, —NR^{j′}C(O)alkynyl, —NR^{j′}C(O)substituted alkynyl, —NR^{j′}C(O)aryl, —NR^{j′}C(O)substituted aryl, —NR^{j′}C(O)heteroaryl, —NR^{j′}C(O)substituted heteroaryl, —NR^{j′}C(O)heterocyclic, and —NR^{j′}C(O)substituted heterocyclic where R^{j′} is hydrogen or alkyl.

“Aminoalkyl” refers to the group amino-alkyl-.

“Aryl” or “Ar” refers to a monovalent aromatic carbocyclic group of from 6 to 14 carbon atoms having a single ring (e.g., phenyl) or multiple condensed rings (e.g., naphthyl or anthryl) which condensed rings may or may not be aromatic (e.g., 2-benzoxazolinone, 2H-1,4-benzoxazin-3(4H)-one-7-yl, and the like) provided that the point of attachment is to an aromatic ring atom. Preferred aryls include phenyl and naphthyl.

“Bicyclic [6,6], [5,6], or [6,5] aryl or heteroaryl group” refers to five or six membered aryl or heteroaryl groups that share a common ring bond with another five or six membered aryl or heteroaryl group to form a fused bicyclic structure having a total of 10 (in the case of [6,6] groups) or 9 (in the case of [5,6] and [6,5] groups) ring atoms.

“Substituted bicyclic [6,6], [5,6], or [6,5] aryl or heteroaryl group” refers to bicyclic [6,6], [5,6], or [6,5] aryl or heteroaryl groups which are substituted with from 1 to 3 substituents, and preferably 1 to 2 substituents, selected from the group consisting of hydroxyl, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxy, carboxy ester, cyano, thiol, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Substituted aryl” refers to aryl groups which are substituted with from 1 to 3 substituents, and preferably 1 to 2 substituents, selected from the group consisting of hydroxyl, acyl, acylamino, acyloxy, alkyl, substituted alkyl, alkoxy, substituted alkoxy, alkenyl, substituted alkenyl, alkynyl, substituted alkynyl, amino, substituted amino, aminoacyl, aryl, substituted aryl, aryloxy, substituted aryloxy, cycloalkoxy, substituted cycloalkoxy, carboxy, carboxy ester, cyano, thiol, cycloalkyl, substituted cycloalkyl, halo, nitro, heteroaryl, substituted heteroaryl, heterocyclic, substituted heterocyclic, heteroaryloxy, substituted heteroaryloxy, heterocyclyloxy, and substituted heterocyclyloxy.

“Aralkyl” or “arylalkyl” refers to the group aryl-alkyl-.

“Arylene” and “substituted arylene” refer to divalent aryl and substituted aryl groups as defined above.

“Aryloxy” refers to the group aryl-O— that includes, by way of example, phenoxy, naphthoxy, and the like.

“Substituted aryloxy” refers to substituted aryl-O— groups.

“Carboxy” or “carboxyl” refers to —COOH or salts thereof.

“Carboxy ester” or “carboxyl ester” refers to the groups —C(O)O-alkyl, —C(O)O-substituted alkyl, —C(O)O-alkenyl, —C(O)O-substituted alkenyl, —C(O)O-alkynyl, —C(O)O-substituted alkynyl, —C(O)O-aryl, —C(O)O-substituted aryl, —C(O)O-heteroaryl, —C(O)O-substituted heteroaryl, —C(O)O-heterocyclic, and —C(O)O-substituted heterocyclic. Preferred carboxy ester are —C(O)O-alkyl, —C(O)O-substituted alkyl, —C(O)O-aryl, and —C(O)O-substituted aryl.

“Cycloalkyl” refers to cyclic alkyl groups of from 3 to 10 carbon atoms having single or multiple cyclic rings optionally comprising 1 to 3 exo carbonyl (C═O) or thiocarbonyl (C═S) groups. Suitable cycloalkyl groups include, by way of example, adamantyl, cyclopropyl, cyclobutyl, cyclopentyl, cyclohexyl, cyclooctyl, 3-oxocyclohexyl, and the like. In multiple condensed rings, one or more of the rings may be other than cycloalkyl (e.g., aryl, heteroaryl or heterocyclic) provided that the point of attachment is to a carbon ring atom of the cycloalkyl group.

“Substituted cycloalkyl” refers to a cycloalkyl group, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxy, carboxy ester, cycloalkyl, substituted cycloalkyl, heteroaryl, substituted heteroaryl, heterocyclic, and substituted heterocyclic. In one embodiment, the cycloalkyl group does not comprise 1 to 3 exo carbonyl or thiocarbonyl groups. In another embodiment, the cycloalkyl group does comprise 1 to 3 exo carbonyl or thiocarbonyl groups. It is understood, that the term “exo” refers to the attachment of a carbonyl or thiocarbonyl to a carbon ring atom of the cycloalkyl group.

“Cycloalkenyl” refers to cyclic alkenyl but not aromatic groups of from 4 to 10 carbon atoms having single or multiple cyclic rings. Suitable cycloalkenyl groups include, by way of example, cyclopentenyl, cyclohexenyl, and cyclooctenyl. In multiple condensed rings, one or more of the rings may be other than cycloalkenyl (e.g., aryl, heteroaryl or heterocyclic) provided that the point of attachment is to a carbon ring atom of the cycloalkyl group.

“Substituted cycloalkenyl” refers to cycloalkenyl groups, having from 1 to 5 substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxy, carboxy ester, cycloalkyl, substituted cycloalkyl, heteroaryl, substituted heteroaryl, heterocyclic, and substituted heterocyclic provided that for hydroxyl substituents the point of attachment is not to a vinyl carbon atom. Substituted cycloalkenyl also refers to cycloalkenyl groups optionally comprising 1 to 3 exo carbonyl or thiocarbonyl groups. It is understood, that the term “exo” refers to the attachment of a carbonyl or thiocarbonyl to a carbon ring atom of the cycloalkenyl group. Suitable groups include, for example, 3-oxocyclohexen-1-yl, and the like. In one embodiment, the cycloalkenyl group does not comprise 1 to 3 exo carbonyl or thiocarbonyl groups. In another embodiment, the cycloalkenyl group does comprise 1 to 3 exo carbonyl or thiocarbonyl groups. Substituted cyclopropyl is a species of substituted cycloalkyl and refers to a C3 cycloalkyl substituted as above.

“Cycloalkylene” and “substituted cycloalkylene” refer to divalent cycloalkyl and substituted cycloalkyl groups as defined above. Preferred cycloalkylene and substituted cycloalkylene groups have three to six carbon atoms.

“Cycloalkenylene” and “substituted cycloalkenylene” refer to divalent cycloalkenyl and substituted cycloalkenyl groups as defined above. Preferred cycloalkenylene and substituted cycloalkenylene groups have four to six carbon atoms.

“Cycloalkoxy” refers to —O-cycloalkyl groups.

“Substituted cycloalkoxy” refers to —O-substituted cycloalkyl groups.

“Halo” or “halogen” refers to fluoro, chloro, bromo and iodo and preferably is fluoro or chloro.

“Haloalkyl” refers to an alkyl group substituted with 1 to 10 halogen atoms.

“Heteroaryl” refers to an aromatic group of from 1 to 15 carbon atoms, preferably from 1 to 10 carbon atoms, and 1 to 4 heteroatoms selected from the group consisting of oxygen, nitrogen and sulfur, within the ring. Such heteroaryl groups can have a single ring (e.g., pyridyl or furyl) or multiple condensed rings (e.g., indolizinyl or benzothienyl). The sulfur atom(s) in the heteroaryl group may optionally be oxidized to sulfoxide and sulfone moieties.

“Substituted heteroaryl” refers to heteroaryl groups that are substituted with from 1 to 3 substituents selected from the same group of substituents defined for substituted aryl.

When a specific heteroaryl is defined as “substituted”, e.g., substituted qunioline, it is understood that such a heteroaryl contains the 1 to 3 substituents as recited above.

“Heteroarylene” and “substituted heteroarylene” refer to divalent heteroaryl and substituted heteroaryl groups as defined above.

“Heteroaryloxy” refers to the group —O-heteroaryl and “substituted heteroaryloxy” refers to the group —O-substituted heteroaryl.

“Heterocycle” or “heterocyclic” refers to a saturated or unsaturated non-aromatic group having a single ring or multiple condensed rings, from 1 to 10 carbon atoms and from 1 to 4 hetero atoms selected from the group consisting of nitrogen, sulfur or oxygen within the ring which ring may optionally comprise 1 to 3 exo carbonyl or thiocarbonyl groups. Preferably, such heterocyclic groups are saturated or unsaturated group having a single ring or multiple condensed rings, from 1 to 10 carbon atoms and from 1 to 4 hetero atoms selected from the group consisting of nitrogen, sulfur, or oxygen within the ring. The sulfur atom(s) in the heteroaryl group may optionally be oxidized to sulfoxide and sulfone moieties.

In multiple condensed rings, one or more of the rings may be other than heterocyclic (e.g., aryl, heteroaryl or cycloalkyl) provided that the point of attachment is to a heterocyclic ring atom. In one embodiment, the heterocyclic group does not comprise 1 to 3 exo carbonyl or thiocarbonyl groups. In a another embodiment, the heterocyclic group does comprise 1 to 3 exo carbonyl or thiocarbonyl groups. It is understood, that the term “exo” refers to the attachment of a carbonyl or thiocarbonyl to a carbon ring atom of the heterocyclic group.

“Substituted heterocyclic” refers to heterocycle groups that are substituted with from 1 to 5 of the same substituents as defined for substituted cycloalkyl. Preferred substituents for substituted heterocyclic groups include heterocyclic groups having from 1 to 3 having substituents selected from the group consisting of alkyl, substituted alkyl, alkoxy, substituted alkoxy, acyl, acylamino, acyloxy, amino, substituted amino, aminoacyl, aryl, substituted aryl, aryloxy, substituted aryloxy, cyano, halogen, hydroxyl, nitro, carboxy, carboxy ester, cycloalkyl, substituted cycloalkyl, heteroaryl, substituted heteroaryl, heterocyclic, and substituted heterocyclic.

When a specific heterocyclic is defined as “substituted”, e.g., substituted morpholino, it is understood that such a heterocycle contains the 1 to 3 substituents as recited above.

Examples of heterocycles and heteroaryls include, but are not limited to, azetidine, pyrrole, imidazole, pyrazole, pyridine, pyrazine, pyrimidine, pyridazine, indolizine, isoindole, indole, dihydroindole, indazole, purine, quinolizine, isoquinoline, quinoline, phthalazine, naphthylpyridine, quinoxaline, quinazoline, cinnoline, carbazole, carboline, phenanthridine, acridine, phenanthroline, isothiazole, phenazine, isoxazole, phenoxazine, phenothiazine, imidazolidine, imidazoline, piperidine, piperazine, indoline, phthalimide, 1,2,3,4-tetrahydro-isoquinoline, 4,5,6,7-tetrahydrobenzo[b]thiophene, thiazole, thiazolidine, thiophene, benzo[b]thiophene, morpholinyl, thiomorpholinyl (also referred to as thiamorpholinyl), piperidinyl, pyrrolidine, tetrahydrofuranyl, and the like.

“Heterocyclyloxy” refers to the group —O-heterocyclic and “substituted heterocyclyloxy” refers to the group —O-substituted heterocyclic.

“Hydroxy” or “hydroxyl” refers to —OH.

“Imino” refers to the group ═NR, where R is hydrogen amino, alkyl, substituted alkyl, aryl, substituted aryl, or hydroxyl.

“Optionally substituted” refers to groups that may be substituted or unsubstituted.

“Sulfonyl” or “Sulfone” refers to the group —SO₂—.

“Thiocarbonyl” refers to the group —C(═S)—.

“Thiol” refers to the group —SH.

“Thioalkyl” refers to the group HS-alkyl-.

The term “amino acid” refers to β-amino acids or to α-amino acids of the formula HR^{b′}N[CH(R^{a′})]_{c′}COOH where R^{a′} is an amino acid side chain, R^{b′} is hydrogen, alkyl, substituted alkyl or aryl and c′ is one or two. Preferably, c′ is one, an α-amino acid, and the α-amino acid is one of the twenty naturally occurring L amino acids.

“Isosteres” are different compounds that have different molecular formulae but exhibit the same or similar properties. For example, tetrazole is an isostere of carboxylic acid because it mimics the properties of carboxylic acid even though they both have very different molecular formulae. Tetrazole is one of many possible isosteric replacements for carboxylic acid. Other carboxylic acid isosteres contemplated by the present invention include —SO₃H, —SO₂NHR^{k′}, —PO₂(R^{k′})₂, —CN, —PO₃(R^{k′})₂, —OR^{k}, —SR^{k′}, —NHCOR^{k′}, —N(R^{k′})₂, —CON(R^{k′})₂, —CONH(O)R^{k′}, —CONHNHSO₂R^{k′}, —COHNSO₂R^{k′}, and —CONR^{k′}CN, where R^{k′} is selected from hydrogen, hydroxyl, halo, haloalkyl, thiocarbonyl, alkoxy, alkenoxy, alkylaryloxy, aryloxy, arylalkyloxy, cyano, nitro, imino, alkylamino, aminoalkyl, thiol, thioalkyl, alkylthio, sulfonyl, alkyl, alkenyl, alkynyl, aryl, aralkyl, cycloalkyl, heteroaryl, heterocycle, and CO₂R^{m′} where R^{m′} is hydrogen, alkyl or alkenyl. In addition, carboxylic acid isosteres can include 5-7 membered carbocycles or heterocycles containing any combination of CH₂, O, S, or N in any chemically stable oxidation state, where any of the atoms of said ring structure are optionally substituted in one or more positions. The following structures are non-limiting examples of preferred carboxylic acid isosteres contemplated by this invention.

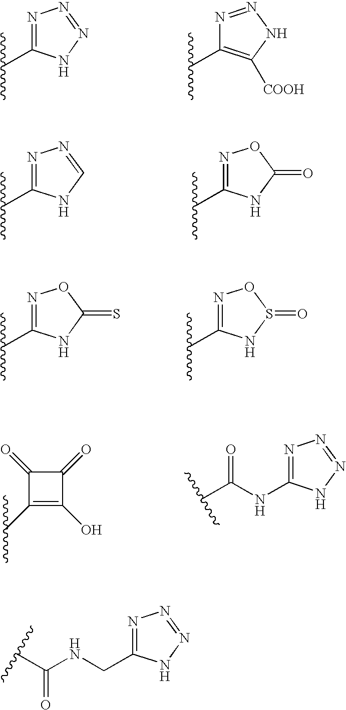

“Carboxylic acid bioisosteres” are compounds that behave as isosteres of carboxylic acids under biological conditions.

Other carboxylic acid isosteres not specifically exemplified or described in this specification are also contemplated by the present invention.

“Pharmaceutically acceptable salt” refers to pharmaceutically acceptable salts of a compound, which salts are derived from a variety of organic and inorganic counter ions well known in the art and include, by way of example only, sodium, potassium, calcium, magnesium, ammonium, tetraalkylammonium, and the like; and when the molecule contains a basic functionality, salts of organic or inorganic acids, such as hydrochloride, hydrobromide, tartrate, mesylate, acetate, maleate, oxalate and the like.

“Prodrug” refers to any derivative of a compound of this invention that is capable of directly or indirectly providing a compound of this invention or an active metabolite or residue thereof when administered to a subject. Particularly favored derivatives and prodrugs are those that increase the bioavailability of the compounds of this invention when such compounds are administered to a subject (e.g., by allowing an orally administered compound to be more readily absorbed into the blood) or which enhance delivery of the parent compound to a biological compartment (e.g., the brain or lymphatic system) relative to the parent species. Prodrugs include ester forms of the compounds of the invention. Examples of ester prodrugs include formate, acetate, propionate, butyrate, acrylate, and ethylsuccinate derivatives. An general overview of prodrugs is provided in T. Higuchi and V. Stella, Pro-drugs as Novel Delivery Systems, Vol. 14 of the A.C.S. Symposium Series, and in Edward B. Roche, ed., Bioreversible Carriers in Drug Design, American Pharmaceutical Association and Pergamon Press, 1987, both of which are incorporated herein by reference.

“Stereoisomer” and “stereoisomers” of a compound refers its enantiomers and diastereomers, and also includes the cis and trans forms of the double bonds present in the compound.

“Tautomer” refers to alternate forms of a molecule that arises from a shift in the position of a proton, such as enol-keto and imine-enamine tautomers, or the tautomeric forms of imidazole.

It is understood that in all substituted groups defined above, polymers arrived at by defining substituents with further substituents to themselves (e.g., substituted aryl having a substituted aryl group as a substituent which is itself substituted with a substituted aryl group, etc.) are not intended for inclusion herein. In such cases, the maximum number of such substituents is three. That is to say that each of the above definitions is constrained by a limitation that, for example, substituted aryl groups are limited to—substituted aryl-(substituted aryl)-substituted aryl.

Similarly, it is understood that the above definitions are not intended to include impermissible substitution patterns (e.g., methyl substituted with five fluoro groups). Such impermissible substitution patterns are well known to the skilled artisan.

General Synthetic Methods

The compounds of this invention can be prepared from readily available starting materials using the following general methods and procedures. It will be appreciated that where typical or preferred process conditions (i.e., reaction temperatures, times, mole ratios of reactants, solvents, pressures, etc.) are given, other process conditions can also be used unless otherwise stated. Optimum reaction conditions may vary with the particular reactants or solvent used, but such conditions can be determined by one skilled in the art by routine optimization procedures.

Additionally, as will be apparent to those skilled in the art, conventional protecting groups may be necessary to prevent certain functional groups from undergoing undesired reactions. Suitable protecting groups for various functional groups as well as suitable conditions for protecting and deprotecting particular functional groups are well known in the art. For example, numerous protecting groups are described in T. W. Greene and P. G. M. Wuts, Protecting Groups in Organic Synthesis, Third Edition, Wiley, New York, 1999, and references cited therein.

If the compounds of this invention contain one or more chiral centers, such compounds can be prepared or isolated as pure stereoisomers, i.e., as individual enantiomers or diastereomers, or as stereoisomer-enriched mixtures. All such stereoisomers (and enriched mixtures) are included within the scope of this invention, unless otherwise indicated. Pure stereoisomers (or enriched mixtures) may be prepared using, for example, optically active starting materials or stereoselective reagents well-known in the art. Alternatively, racemic mixtures of such compounds can be separated using, for example, chiral column chromatography, chiral resolving agents and the like.

Compounds of the invention may generally be prepared in an analogous manner to that shown in Scheme 1 below. It is understood that for illustrative purposes, Scheme 1 employs the following substitution patterns: B, D, and A are CH, R is cyclohexyl; L is a bond; Z is carboxy or carboxy ester; and Ar is substituted quinolin-6-yl. Other compounds and substitution patterns can readily be made by the following the procedures below with proper substitution of reagents. Such factors are well within the skill of the art.

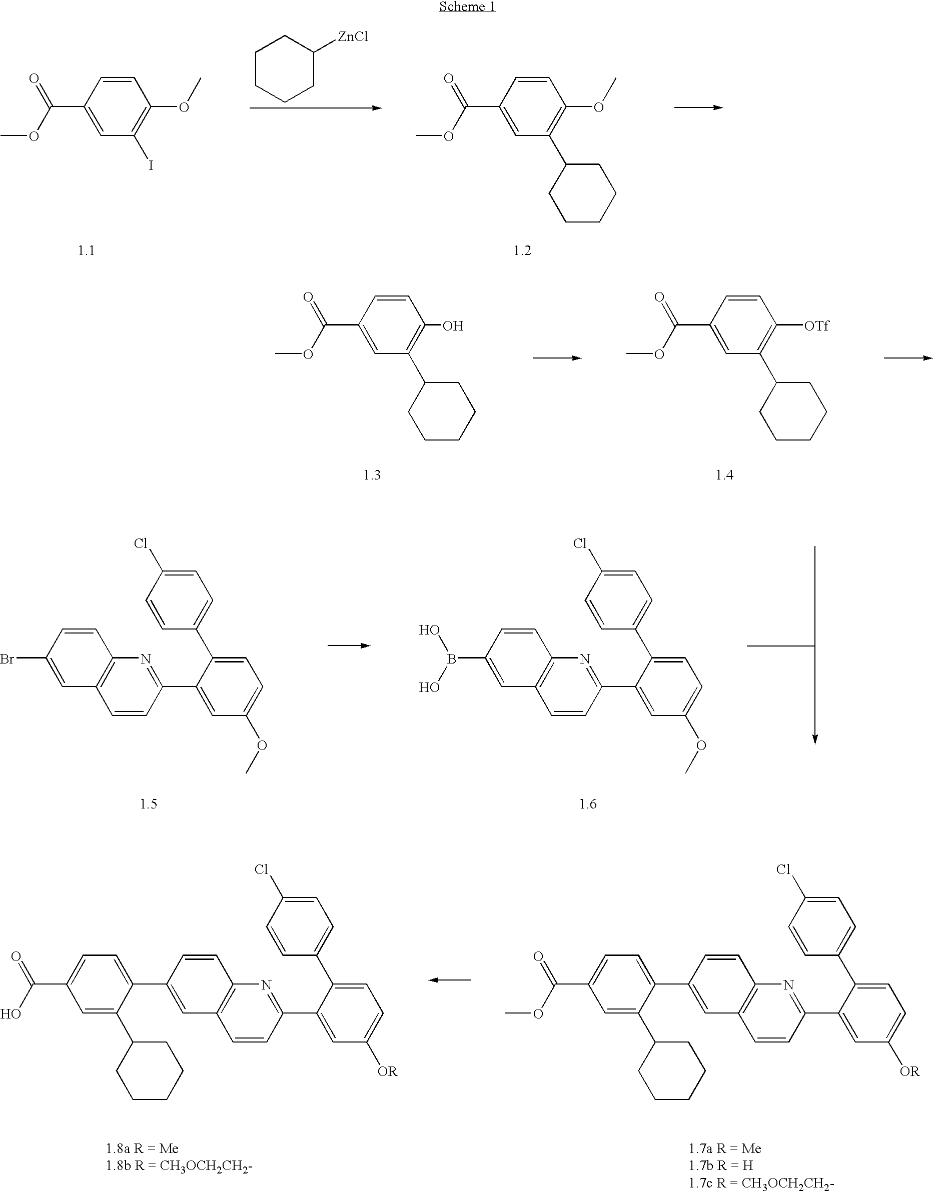

In one aspect, the R and Ar substituents may be introduced onto the 6-membered aromatic core via the respective Negishi and Suzuki coupling reactions as illustrated in Scheme 1 and detailed in Example 3. The cyclohexyl zinc chloride reagent (generated in situ by adding cyclohexyl magnesium chloride to a THF solution of ZnCl₂) is treated with iodide 1.1 in the presence of a metal catalyst such as Pd(P(tBu)₃)₂ and a polar co-solvent such as 1-methyl-2-pyrrolidinone to give compound 1.2 containing the desired cyclohexyl R group.

Methyl ether 1.2 is next converted to a suitably activated Suzuki coupling partner such as triflate 1.4 by demethylation with BBr₃ and treatment of the resulting alcohol 1.3 with triflic anhydride. Suitable Ar Suzuki couping partners are boronic acids such as compound 1.6 and can be synthesized from the corresponding halide such as bromide 1.5 by treatment with bis(neopentylglycolato)diboron and a Pd catalyst such as [P(Ph)₃]₂Pd(II)Cl. Details of the synthesis of Ar halo starting materials such as bromide 1.5 are shown in Examples 1 and 2.

Triflate 1.4 and boronic acid 1.6 are reacted in the presence of a Pd catalyst such as tetrakis(triphenylphosphino)-palladium to form quinoline 1.7a. Functional group manipulations using standard chemical reactions can further be carried out to modify the substituents on the Ar group to give compounds 1.7b and 1.7c. Deprotection of methyl ether 1.7b can be accomplished by reaction of the ether with a Lewis acid such as BBr₃. The resulting alcohol 1.7b is next reacted with a halide such as 2-bromoethyl methyl ether in the presence of a base such as K₂CO₃ in an inert solvent such as DMF. Modification of the Z groups such as saponification of esters 1.7a and 1.7c can be accomplished with a NaOH/MeOH/THF mixture to give acids 1.8a and 1.8b. Details of these reactions can be found in Examples 3 and 4.

Compounds of the invention may also generally be prepared in an analogous manner to that shown in Scheme 2 below. It is understood that for illustrative purposes, Scheme 2 employs the following substitution patterns: D and A are CH, R is cyclohexyl, L¹ is ethylene or ethene, R¹ is carboxy or carboxy derivative, L is a bond, Z is carboxy or carboxy ester, and Ar is 2-(2,4-dimethylthiazol-5-yl)-5-(1-morpholino-1-oxomethyl)-quinolin-6-yl. Other compounds and substitution patterns can readily be made by the following the procedures below with proper substitution of reagents. Such factors are well within the skill of the art.

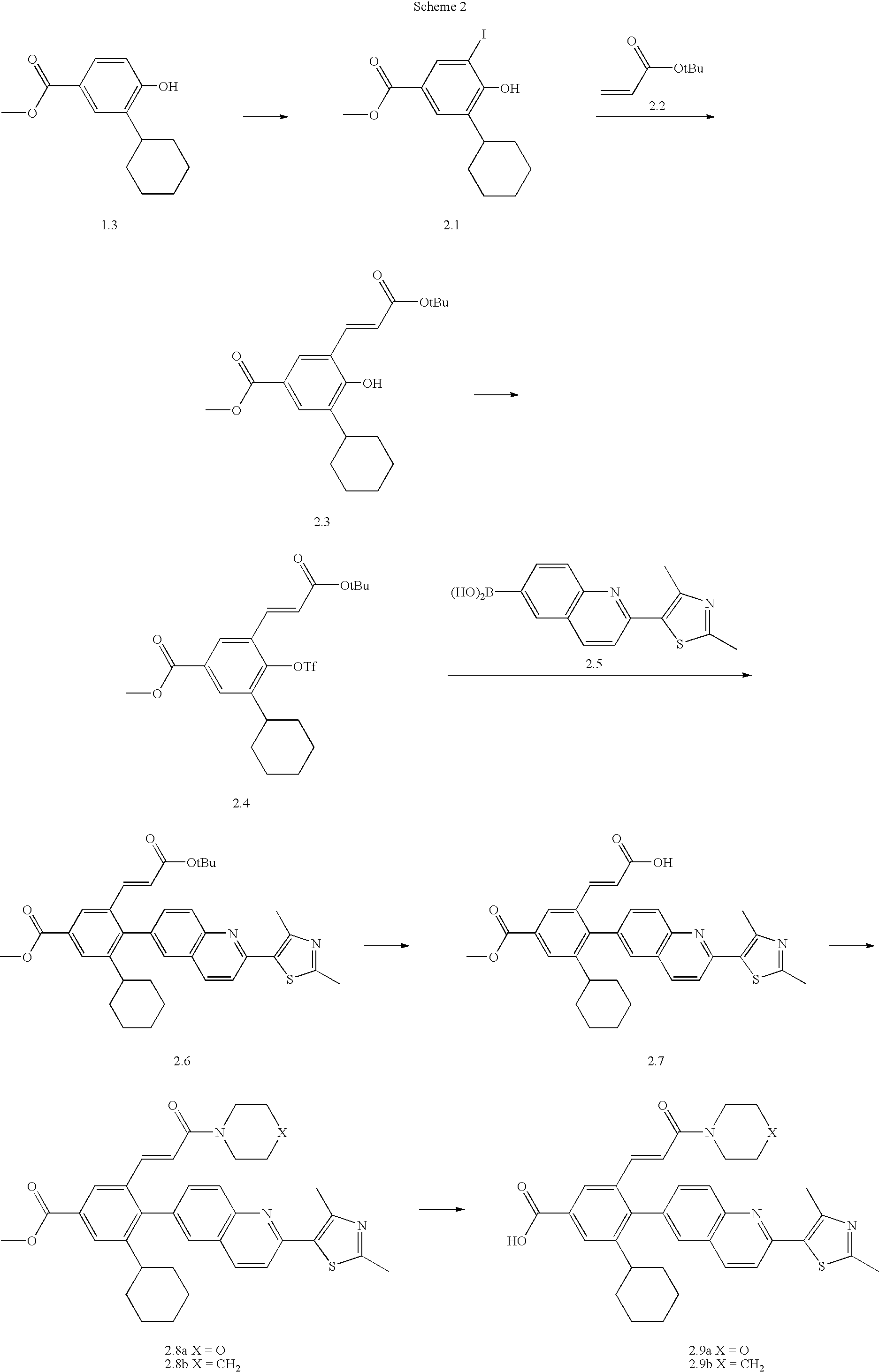

In one aspect, the -L¹-R¹ group may be introduced by first iodinating compound 1.3 with an iodine reagent such as N-iodosuccinimide in a protic solvent such as acetic acid. Iodide 2.1 can be coupled with an -L¹-R¹ group such as tert-butyl acrylate in the presence of (PPh₃)₂PdCl₂ and Et₃N and in a polar solvent such as DMF. The resulting alcohol 2.3 can react with triflic anhydride to form triflate 2.4. An Ar group such as boronic acid 2.5 can next be coupled with triflate 2.4 under standard Suzuki conditions to form ester 2.6. Selective removal of the t-butyl ester 2.6 using an acid such as trifluoroacetic acid gives compound 2.7. Modifications of the R¹ group can occur at this stage such as by coupling acid 2.7 with a heterocycle such as morpholine or piperidine in presence of an amide coupling reagent such as HBTU or HATU and an bulky organic base such as diisopropylethylamine to give compounds 2.8a and 2.8b. Saponification of the esters such as with a NaOH/MeOH/THF mixture gives acids 2.9a and 2.9b where Z is a carboxylic acid. Details of the synthesis of the compounds in Scheme 2 can be found in Examples 5 and 6.

Schemes 3 below illustrates the synthesis of compounds where L¹ is —CH₂—. It is understood that the substitution pattern in Scheme 3 is for illustrative purposes. Other compounds and substitution patterns can readily be made by the following the procedures below with proper substitution of reagents. Such factors are well within the skill of the art.

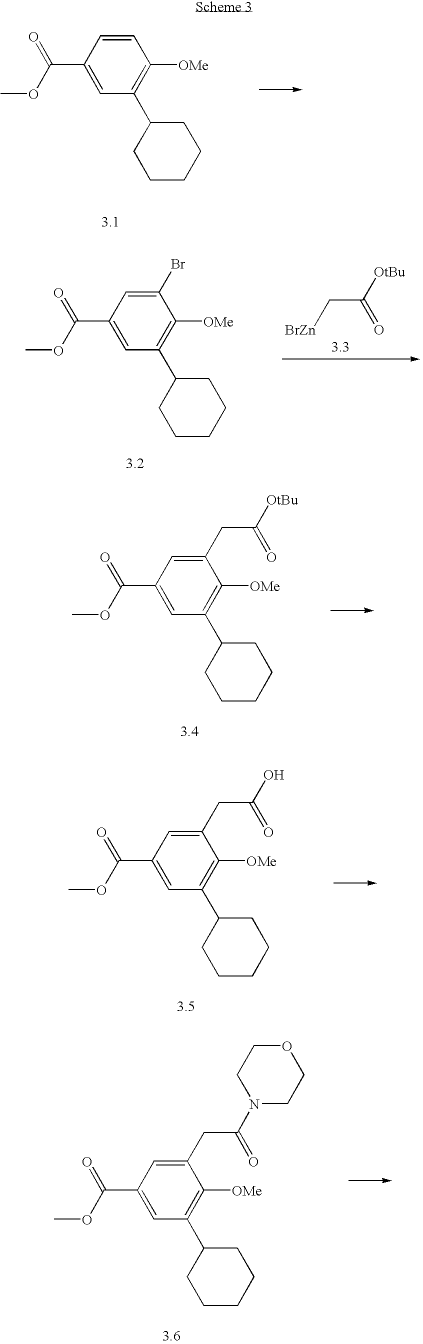

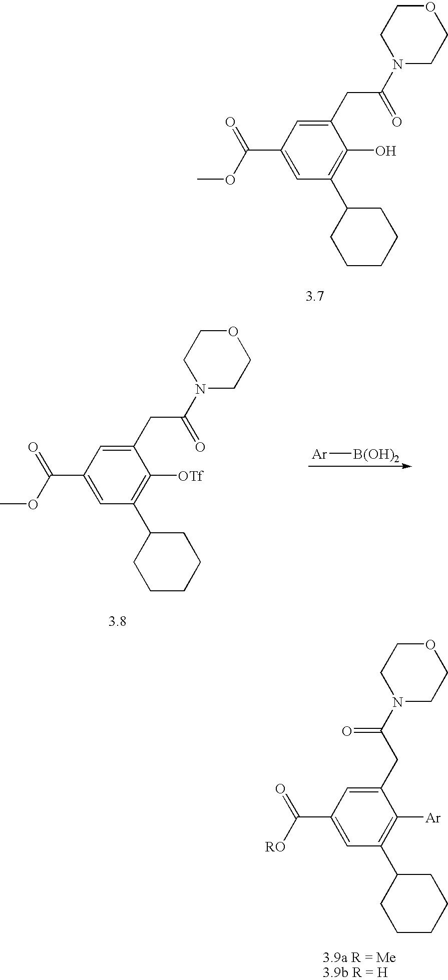

Compound 3.1 can be treated with bromine to form bromide 3.2 that can then be coupled with bromoacetic acid t-butyl ester zincate 3.3 under Negishi coupling conditions such as by reaction with (PPh₃)₂PdCl₂ and Et₃N and in a polar solvent such as DMF. Product ester 3.4 can then be elaborated under conventional conditions as described for Scheme 2 to give compounds 3.9a and 3.9b.

Scheme 4 below illustrates the synthesis of compounds where L¹ is a bond. It is understood that the substitution pattern in Scheme 4 is for illustrative purposes. Other compounds and substitution patterns can readily be made by the following the procedures below with proper substitution of reagents. Such factors are well within the skill of the art.

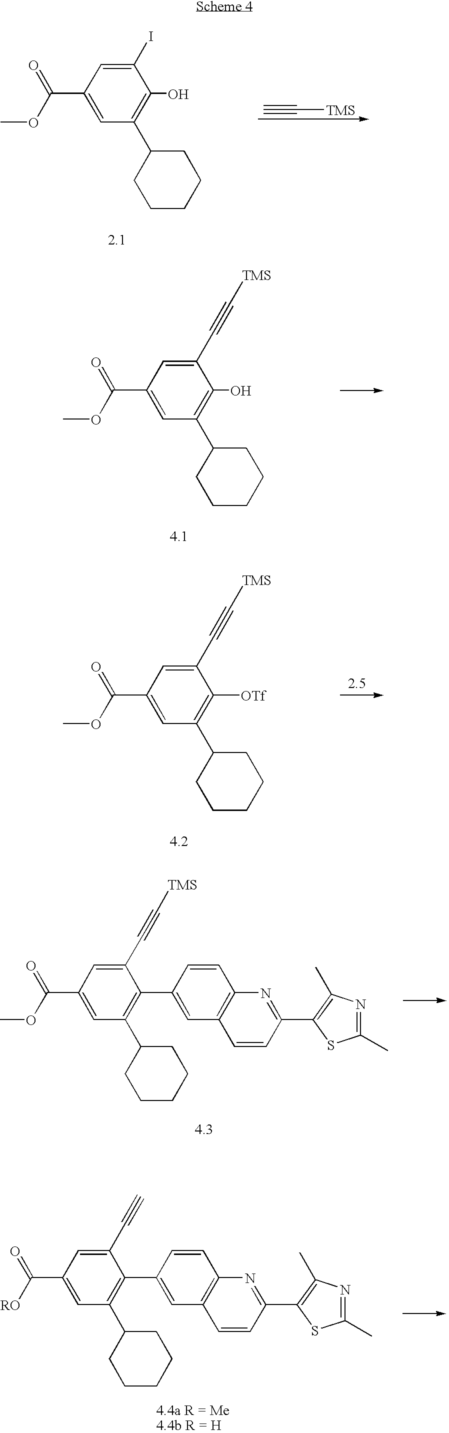

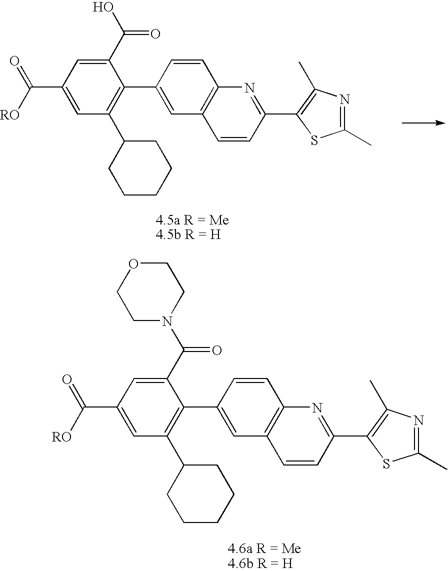

Iodide 2.1 is exposed to CuI, (PPh₃)₄Pd, Et₃N (trimethylsilyl)acetylene in a polar solvent such as DMF to form acetylene 4.1. The alcohol moiety is next reacted with triflic anhydride to yield 4.2 can be coupled with boronic acid 2.5 give silane 4.3. Removal of both the silyl group and the methyl ester can be effected with base such as by treatment of 4.3 with NaOH in methanol to give acid 4.4b. The silyl group can also selectively be removed with K₂CO₃ to give ester 4.4a. Exposure of 4.4a or 4.4b to a CH₂Cl₂/tert-BuOH containing MeReO₃ and 50% aq. H₂O₂ gives acid 4.5a and 4.5b. Addition of the R¹ heterocycle morpholine can be effected by treating 4.5a with morpholine under a variety of amide coupling conditions such as with HBTU or HATU and diisopropylethylamine. Details of this reaction sequence can be found in Examples 7-9.

Scheme 5 below illustrates the synthesis of compounds where L is —CH₂—. It is understood that the substitution pattern in Scheme 5 is for illustrative purposes. Other compounds and substitution patterns can readily be made by the following the procedures below with proper substitution of reagents. Such factors are well within the skill of the art.

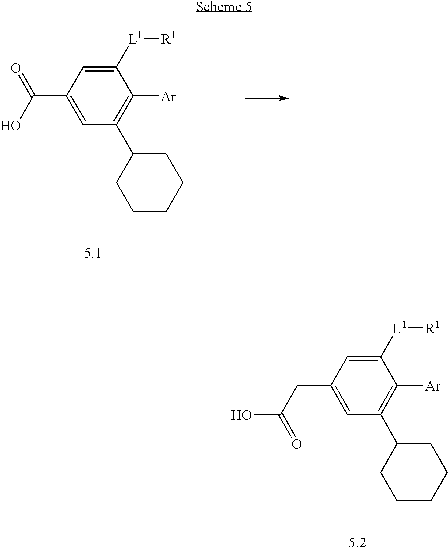

Acid 5.1 can be converted to acid 5.2 with via the Arndt-Eistert reaction. The acid moiety is treated with thionyl chloride to form the corresponding acid chloride that is next reacted with diazomethane. Exposure of the product diazo ketone to Ag₂O and H₂O and heating the resulting solution gives the homologated acid 5.2.

Scheme 6 below illustrates the synthesis of compounds where L is —CH═CH—. It is understood that the substitution pattern in Scheme 6 is for illustrative purposes. Other compounds and substitution patterns can readily be made by the following the procedures below with proper substitution of reagents. Such factors are well within the skill of the art.

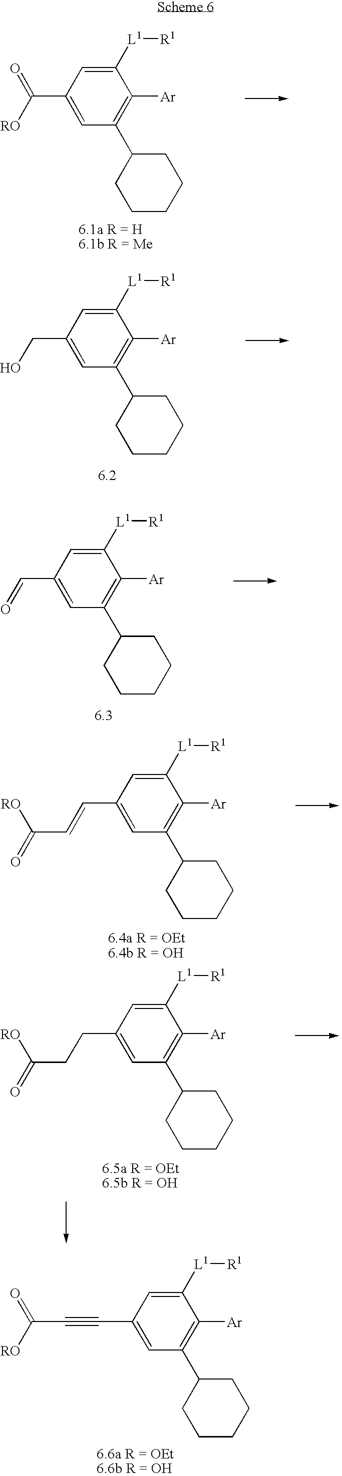

Acid 6.1a or ester 6.1b can be reduced to alcohol 6.2 with BH₃/THF or LiBH₄ respectively. Alcohol 6.2 can be oxidized to aldehyde 6.3 using an oxidizing agent such as MnO₂ or N-methylmorpholine N-oxide/tetra-n-propylammonium perrruthenate(VII). Coupling of the aldehyde 6.3 with Horner Emmons reagent triethylphosphonoacetate in the presence of an organic base such as DBU (1,8-diazabicyclo[5.4.0]undec-7-ene) and a salt such as LiCl affords ester 6.4a that can also be hydrolyzed to acid 6.4b. Hydrogenation of 6.4a or 6.4b with H₂ over palladium gives compounds 6.5a and 6.5b. Alternatively treatment of 6.4a or 6.4b with Br₂ followed by a subsequent reaction with a suitable base such as potassium t-butoxide provides for compounds 6.6a and 6.6b.

Administration and Pharmaceutical Composition

The present invention provides novel compounds possessing antiviral activity, including Flaviviridae family viruses such as hepatitis C virus. The compounds of this invention inhibit viral replication by inhibiting the enzymes involved in replication, including RNA dependent RNA polymerase. They may also inhibit other enzymes utilized in the activity or proliferation of Flaviviridae viruses.

Compounds of this invention maybe used alone or in combination with other compounds to treat viruses.

In general, the compounds of this invention will be administered in a therapeutically effective amount by any of the accepted modes of administration for agents that serve similar utilities. The actual amount of the compound of this invention, i.e., the active ingredient, will depend upon numerous factors such as the severity of the disease to be treated, the age and relative health of the subject, the potency of the compound used, the route and form of administration, and other factors. The drug can be administered more than once a day, preferably once or twice a day.

Therapeutically effective amounts of compounds of the present invention may range from approximately 0.01 to 50 mg per kilogram body weight of the recipient per day; preferably about 0.1-25 mg/kg/day, more preferably from about 0.1 to 10 mg/kg/day. Thus, for administration to a 70 kg person, the dosage range would most preferably be about 7-700 mg per day.

In general, compounds of this invention will be administered as pharmaceutical compositions by any one of the following routes: oral, systemic (e.g., transdermal, intranasal or by suppository), or parenteral (e.g., intramuscular, intravenous or subcutaneous) administration. The preferred manner of administration is oral using a convenient daily dosage regimen that can be adjusted according to the degree of affliction. Compositions can take the form of tablets, pills, capsules, semisolids, powders, sustained release formulations, solutions, suspensions, elixirs, aerosols, or any other appropriate compositions. Another preferred manner for administering compounds of this invention is inhalation.

The choice of formulation depends on various factors such as the mode of drug administration and bioavailability of the drug substance. For delivery via inhalation the compound can be formulated as liquid solution, suspensions, aerosol propellants or dry powder and loaded into a suitable dispenser for administration. There are several types of pharmaceutical inhalation devices-nebulizer inhalers, metered dose inhalers (MDI) and dry powder inhalers (DPI). Nebulizer devices produce a stream of high velocity air that causes the therapeutic agents (which are formulated in a liquid form) to spray as a mist that is carried into the patient's respiratory tract. MDI's typically are formulation packaged with a compressed gas. Upon actuation, the device discharges a measured amount of therapeutic agent by compressed gas, thus affording a reliable method of administering a set amount of agent. DPI dispenses therapeutic agents in the form of a free flowing powder that can be dispersed in the patient's inspiratory air-stream during breathing by the device. In order to achieve a free flowing powder, the therapeutic agent is formulated with an excipient such as lactose. A measured amount of the therapeutic agent is stored in a capsule form and is dispensed with each actuation.

Recently, pharmaceutical formulations have been developed especially for drugs that show poor bioavailability based upon the principle that bioavailability can be increased by increasing the surface area i.e., decreasing particle size. For example, U.S. Pat. No. 4,107,288 describes a pharmaceutical formulation having particles in the size range from 10 to 1,000 nm in which the active material is supported on a crosslinked matrix of macromolecules. U.S. Pat. No. 5,145,684 describes the production of a pharmaceutical formulation in which the drug substance is pulverized to nanoparticles (average particle size of 400 nm) in the presence of a surface modifier and then dispersed in a liquid medium to give a pharmaceutical formulation that exhibits remarkably high bioavailability.

The compositions are comprised of, in general, a compound of the present invention in combination with at least one pharmaceutically acceptable excipient. Acceptable excipients are non-toxic, aid administration, and do not adversely affect the therapeutic benefit of the claimed compounds. Such excipient may be any solid, liquid, semi-solid or, in the case of an aerosol composition, gaseous excipient that is generally available to one of skill in the art.

Solid pharmaceutical excipients include starch, cellulose, talc, glucose, lactose, sucrose, gelatin, malt, rice, flour, chalk, silica gel, magnesium stearate, sodium stearate, glycerol monostearate, sodium chloride, dried skim milk and the like. Liquid and semisolid excipients may be selected from glycerol, propylene glycol, water, ethanol and various oils, including those of petroleum, animal, vegetable or synthetic origin, e.g., peanut oil, soybean oil, mineral oil, sesame oil, etc. Preferred liquid carriers, particularly for injectable solutions, include water, saline, aqueous dextrose, and glycols.

Compressed gases may be used to disperse a compound of this invention in aerosol form. Inert gases suitable for this purpose are nitrogen, carbon dioxide, etc. Other suitable pharmaceutical excipients and their formulations are described in Remington's Pharmaceutical Sciences, edited by E. W. Martin (Mack Publishing Company, 18th ed., 1990).

The amount of the compound in a formulation can vary within the full range employed by those skilled in the art. Typically, the formulation will contain, on a weight percent (wt %) basis, from about 0.01-99.99 wt % of a compound of the present invention based on the total formulation, with the balance being one or more suitable pharmaceutical excipients. Preferably, the compound is present at a level of about 1-80 wt %. Representative pharmaceutical formulations are described below.

Additionally, the present invention is directed to a pharmaceutical composition comprising a therapeutically effective amount of a compound of the present invention in combination with a therapeutically effective amount of another active agent against RNA-dependent RNA virus and, in particular, against HCV. Agents active against HCV include, but are not limited to, ribavirin, levovirin, viramidine, thymosin alpha-1, an inhibitor of HCV NS3 serine protease, or an inhibitor of inosine monophosphate dehydrognease, interferon-α, pegylated interferon-α (peginterferon-α), a combination of interferon-α and ribavirin, a combination of peginterferon-α and ribavirin, a combination of interferon-α and levovirin, and a combination of peginterferon-α and levovirin. Interferon-α includes, but is not limited to, recombinant interferon-α2a (such as ROFERON interferon available from Hoffman-LaRoche, Nutley, N.J.), interferon-α2b (such as Intron-A interferon available from Schering Corp., Kenilworth, N.J., USA), a consensus interferon, and a purified interferon-α product. For a discussion of ribavirin and its activity against HCV, see J. O. Saunders and S. A. Raybuck, “Inosine Monophosphate Dehydrogenase: Consideration of Structure, Kinetics and Therapeutic Potential,” Ann. Rep. Med. Chem., 35:201-210 (2000).

The agents active against hepatitis C virus also include agents that inhibit HCV proteases, HCV polymerase, HCV helicase, HCV NS4B protein, HCV entry, HCV assembly, HCV egress, HCV NS5A protein, and inosine 5′-monophosphate dehydrogenase. Other agents include nucleoside analogs for the treatment of an HCV infection. Still other compounds include those disclosed in WO 2004/014313 and WO 2004/014852 and in the references cited therein. The patent applications WO 2004/014313 and WO 2004/014852 are hereby incorporated by references in their entirety.

Specific antiviral agents include Omega IFN (BioMedicines Inc.), BILN-2061 (Boehringer Ingelheim), Summetrel (Endo Pharmaceuticals Holdings Inc.), Roferon A (F. Hoffman-La Roche), Pegasys (F. Hoffman-La Roche), Pegasys/Ribaravin (F. Hoffman-La Roche), CellCept (F. Hoffman-La Roche), Wellferon (GlaxoSmithiKline), Albuferon-a (Human Genome Sciences Inc.), Levovirin (ICN Pharmaceuticals), IDN-6556 (Idun Pharmaceuticals), IP-501 (Indevus Pharmaceuticals), Actimmune (InterMune Inc.), Infergen A (InterMune Inc.), ISIS 14803 (ISIS Pharamceuticals Inc.), JTK-003 (Japan Tobacco Inc.), Pegasys/Ceplene (Maxim Pharmaceuticals), Ceplene (Maxim Pharmaceuticals), Civacir (Nabi Biopharmaceuticals Inc.), Intron A/Zadaxin (RegeneRx), Levovirin (Ribapharm Inc.), Viramidine(Ribapharm Inc.), Heptazyme (Ribozyme Pharmaceuticals), Intron A (Schering-Plough), PEG-Intron (Schering-Plough), Rebetron (Schering-Plough), Ribavirin (Schering-Plough), PEG-Intron/Ribavirin (Schering-Plough), Zadazim (SciClone), Rebif (Serono), IFN-b/EMZ701 (Transition Therapeutics), T67 (Tularik Inc.), VX-497 (Vertex Pharmaceuticals Inc.), VX-950/LY-570310 (Vertex Pharmaceuticals Inc.), Omniferon (Viragen Inc.), XTL-002 (XTL Biopharmaceuticals), SCH 503034 (Schering-Plough), isatoribine and its prodrugs ANA971 and ANA975 (Anadys), R1479 (Roche Biosciences), Valopicitabine (Idenix), NIM811 (Novartis), and Actilon (Coley Pharmaceuticals).

In some embodiments, the compositions and methods of the present invention contain a compound of formula 1 and interferon. In some aspects, the interferon is selected from the group consisting of interferon alpha 2B, pegylated interferon alpha, consensus interferon, interferon alpha 2A, and lymphoblastiod interferon tau.

In other embodiments the compositions and methods of the present invention contain a compound of formula 1 and a compound having anti-HCV activity is selected from the group consisting of interleukin 2, interleukin 6, interleukin 12, a compound that enhances the development of a type 1 helper T cell response, interfering RNA, anti-sense RNA, Imiqimod, ribavirin, an inosine 5′monophospate dehydrogenase inhibitor, amantadine, and rimantadine.

### FORMULATION EXAMPLES

The following are representative pharmaceutical formulations containing a compound of formula I.

### Formulation Example 1

### Tablet Formulation

The following ingredients are mixed intimately and pressed into single scored tablets.

| | Quantity per |
|---|---|
| Ingredient | tablet, mg |
| compound of this invention | 400 |
| cornstarch | 50 |
| croscarmellose sodium | 25 |
| lactose | 120 |
| magnesium stearate | 5 |

### Formulation Example 2

### Capsule Formulation

The following ingredients are mixed intimately and loaded into a hard-shell gelatin capsule.

| | Quantity per |
|---|---|
| Ingredient | capsule, mg |
| compound of this invention | 200 |
| lactose, spray-dried | 148 |
| magnesium stearate | 2 |

### Formulation Example 3

### Suspension Formulation

The following ingredients are mixed to form a suspension for oral administration (q.s.=sufficient amount).

| Ingredient | Amount | |
|---|---|---|
| compound of this invention | 1.0 | g |
| fumaric acid | 0.5 | g |
| sodium chloride | 2.0 | g |
| methyl paraben | 0.15 | g |
| propyl paraben | 0.05 | g |
| granulated sugar | 25.0 | g |
| sorbitol (70% solution) | 13.00 | g |
| Veegum K (Vanderbilt Co.) | 1.0 | g |
| flavoring | 0.035 | mL |
| colorings | 0.5 | mg |
| distilled water | q.s. to 100 | mL |

### Formulation Example 4

### Injectable Formulation

The following ingredients are mixed to form an injectable formulation.

| Ingredient | Amount | |
|---|---|---|
| compound of this invention | 0.2 mg-20 | mg |
| sodium acetate buffer solution, 0.4 M | 2.0 | mL |
| HCl (1N) or NaOH (1N) | q.s. to suitable pH | |
| water (distilled, sterile) | q.s. to 20 | mL |

### Formulation Example 5

### Suppository Formulation

A suppository of total weight 2.5 g is prepared by mixing the compound of the invention with Witepsol® H-15 (triglycerides of saturated vegetable fatty acid; Riches-Nelson, Inc., New York), and has the following composition:

| Ingredient | Amount |
|---|---|
| Compound of the invention | 500 mg |
| Witepsol ® H-15 | balance |

In the examples below and the synthetic schemes above, the following abbreviations have the following meanings. If an abbreviation is not defined, it has its generally accepted meaning.
μL=microliters
μM=micromolar
μg=micrograms
aq.=aqueous
AcOH=acetic acid
boc=t-butoxycarbonyl
br=broad
d=doublet
δ=chemical shift
DCM=dichloromethane
dd=doublet of doublets
DIEA=diisopropylethylamine
DMAP=4-N,N-dimethylaminopyridine
DMEM=Dulbeco's Modified Eagle's Medium
DMF=N,N-dimethylformamide
DMSO=dimethylsulfoxide
DTT=dithiothreotol
EDTA=ethylenediaminetetraacetic acid
eq.=equivalent
ESI=electrospray ionization
EtOAc=ethyl acetate
g=gram
h or hr=hours
HATU=O-(7-Azabenzotriazol-1-yl)-N,N,N′,N′-tetramethyluronium hexafluorophosphate
HBTU=O-Benzotriazol-1-yl-N,N,N′,N′-tetramethyluronium hexafluorophosphate
HCV=hepatitus C virus
HPLC=high performance liquid chromatography
Hz=hertz
IPTG=isopropyl-β-D-thiogalactopyranoside
IU=International Units
IC₅₀=inhibitory concentration at 50% inhibition
J=coupling constant (given in Hz unless otherwise indicated)
N=Normal
ng=nanogram
NIS=N-iodosuccinimide
nm=nanometer
nM=nanomolar
NMP=1-methyl-2-pyrrolidinone
NMR=nuclear magnetic resonance
NTA=nitrilotriacetic acid
NTP=nucleoside triphosphate
m=multiplet
M=molar
M+H⁺=parent mass spectrum peak plus H⁺
MeOH=methanol
mg=milligram
min.=minutes
mL=milliliter
mM=millimolar
mmol=millimole
MS=mass spectrum
PCR=Polymerase chain reaction
ppm=parts per million
psi=pounds per square inch
RP-HPLC=reversed phase high performance liquid chromatography
s=singlet
t=triplet
tBu=tert-butyl
TC₅₀=Toxic concentration at 50% cell toxicity
tetrakis or tetrakis=tetrakis(triphenylphosphine)palladium(0) palladium
Tf₂O=triflic anhydride
TFA=trifluoroacetic acid
THF=tetrahydrofuran
Tris=Tris(hydroxymenthyl)aminomethane
UTP=uridine triphosphate

### SYNTHETIC EXAMPLES

### Example 1

### Synthesis of 6-bromo-2-(4′-chloro-4-methoxy-biphenyl-2-yl)-quinoline 1.5

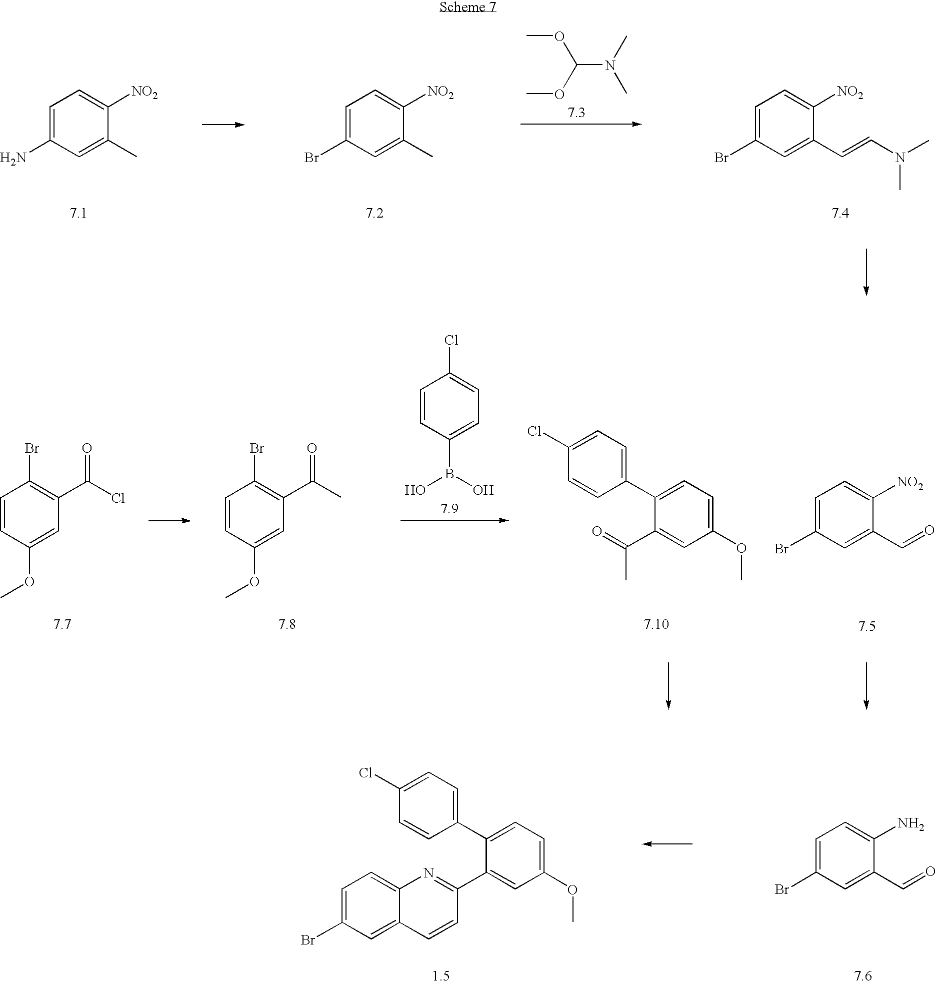

### Preparation of 4-bromo-2-methyl-1-nitro-benzene 7.2

To an ice cold solution of 10.0 g (65.7 mmol) 3-methyl-4-nitro-phenylamine in 200 mL acetone, was added 21 mL (197.2 mmol) 48% HBr. 4.54 g (65.7 mmol) NaNO₂ was dissolved in 20 mL water and was added dropwise to the amine solution at a rate to keep the temperature under 5° C. The mixture was stirred at this temperature for an additional 10 minutes then 1.5 g (10 mmol) solid CuBr was added portion-wise at a rate to keep the temperature under 15° C. The reaction was complete when no more nitrogen evaluated (about 15 minutes). The reaction mixture was evaporated to dryness; the residue was dissolved in a mixture of 500 mL water and 750 mL ethyl acetate. The organic phase was separated, washed with water (2×), saturated NaCl (2×) and was dried (Na₂SO₄). It was then evaporated to dryness to give the crude product as a yellow solid which was purified by filtering through 400 mL silica gel pad using toluene elution;

Yield: 10.45 g (73%);

¹H-NMR (CDCl₃): δ (ppm) 7.87 (d, 1H, J=8.7 Hz), 7.51-7.46 (m, 2H), 2.61 (s, 3H).

### Preparation of [(E)-2-(5-bromo-2-nitro-phenyl)-vinyl]-dimethyl-amine 7.4:

A mixture of 9.26 g (42.9 mmol) of compound 7.2, 14.3 mL (107.2 mmol) N,N-dimethylformamide dimethylacetal 7.3 and 11 mL DMF was heated under a slow argon flow at 145° C. (bath) for two hours. The reaction mixture was then evaporated to dryness. The dark pink product crystallized upon standing;

MS: 271.01 & 273.01 (M+H⁺);

¹H-NMR (DMSO-d₆): δ (ppm) 7.88 (d, 1H), 7.68 (dd, 1H), 7.58 (d, 1H), 7.05 (d, 1H), 5.59 (d, 1H), 2.90 (s, 6H).

### Preparation of 5-bromo-2-nitro-benzaldehyde 7.5

Compound 7.4 (11.63 g (42.9 mmol)) was dissolved in 500 mL 1:1 mixture of THF and water. To this solution 34.3 g (160 mmol) NaIO₄ was added and the mixture was stirred at room temperature for 1 hr while the dark solution became pale yellow with a heavy precipitate. The solid material was filtered off, washed twice with 100 mL ethyl acetate and the organic phases were pooled and evaporated to dryness. The residue was filtered through a 400 mL silica gel pad using toluene for elution to get 7.08 g (71%) of the title compound;

H¹-NMR (DMSO-d₆): δ(ppm) 10.10 (s, 1H), 8.09-7.99 (m, 3H).

### Preparation of 2-amino-5-bromo-benzaldehyde 7.6

Compound 7.5 was synthesized from 5.45 g (23.7 mmol) of compound 5 using the procedure of L. I. Smith and J. W. Opie (Org. Synth. Coll. Vol. 3, 56) in 55% yield(2.6 g);

MS: 199.97 & 201.97 (M+H⁺);

H¹-NMR (CDCl₃): δ (ppm) 9.75 (s, 1H), 7.71 (s, 1H), 7.39 (d, 1H, J=9.3 Hz), 7.22 (s, 2H), 6.72 (d, 1H, J=9.3 Hz).

### Preparation of 1-(2-bromo-5-methoxy-phenyl)-ethanone 7.8

To an ice cold solution of 8.75 g (35 mmol) 2-bromo-5-methoxy-benzoyl chloride in 40 mL toluene, 9.63 mL (19.25 mmol) of a 2M toluene solution of dimethylzinc was added under argon atmosphere (dimethylzinc is pyrophoric—contact with air should be avoided). The ice bath was removed and the mixture slowly warmed up to room temperature. Once the reaction starts it proceeds rapidly resulting in a turbid solution. The reaction was complete in 30 minutes. It was then cooled back to 0° C. and was quenched by adding 10 mL ethanol. The mixture was evaporated to dryness, the residue was dissolved in a mixture of 50 mL 1M HCl and 100 mL ethyl acetate. The organic phase was separated and washed with 50 mL water (2×), brine (2×) and was dried (Na₂SO₄). The final solution was evaporated and the oil dried overnight in high vacuum to give 7.96 g (99%) of the title compound as a colorless liquid;

¹H-NMR (CDCl₃): δ (ppm) 7.46 (d, 1H), 6.96 (d, 1H), 6.83 (dd, 1H), 3.80 (s, 3H), 2.63 (s, 3H).

### Preparation of 1-(4′-chloro-4-methoxy-biphenyl-2-yl)-ethanone 7.10

A mixture of compound 7.8 (6.0 g, 26.19 mmol), 4-chlorobenzeneboronic acid (4.51 g, 28.81 mmol) and Pd(PPh₃)₄ (0.303 g, 0.262 mmol) in toluene (250 mL), MeOH (60 mL) and 2 M NaHCO₃ (25 mL) was stirred under argon at 80° C. for 16 h. After removal of the solvent, the dry residue was dissolved in CHCl₃ (150 mL) and filtered. The solvent was evaporated and the residue was purified by chromatography using CHCl₃-MeOH (70:1) as eluent to give the title compound (6.33 g, 93%);

¹H NMR (CDCl₃): 7.36 (d, 2H, J=8.4 Hz), 7.27-7.21 (m, 4H), 7.02 (d, 1H, J=2.7 Hz), 3.86 (s, 3H), 2.05 (s, 3H). MS (ESI) 261.07 (M+H).

### Preparation of 6-bromo-2-(4′-chloro-4-methoxy-biphenyl-2-yl)-quinoline 1.5

Compound 7.10 (100 mg (0.5 mmol)) and compound 7.6 (130 mg (0.5 mmol)) were dissolved in 5 mL ethanol and 800 μL 10% KOH (1.5 mmol) was added and the mixture was kept in a 90° C. bath under argon overnight. The solvent was evaporated and the residue triturated with water. The semi solid compound 1.5 was purified on a 400 mL silica gel pad using toluene for elution to give 2.03 g (44%) yellow gummy material;

MS: 424.03 & 426.03 (M+H⁺);

¹H-NMR (DMSO-d₆): δ (ppm) 8.20 (d, 1H, J=2.1 Hz), 8.10 (d, 1H, J=9.0 Hz), 7.93-7.83 (m, 2H), 7.40 (d, 1H, J=8.4 Hz), 7.26-7.23 (m, 3H0, 7.16-7.03 (m, 4H), 3.85 (s, 3H).

### Example 2

### Synthesis of 2-(2,4-dimethyl-thiazol-5-yl)-quinoline-6-boronic acid 2.5

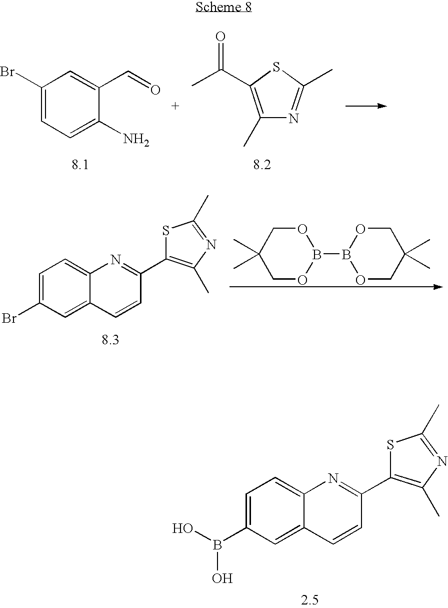

A mixture of 2-amino-5-bromobenzaldehyde (1.071 g, 5.354 mmol), 5-acetyl-2,4-dimethylthiazole (723 μL, 5.354 mmol) and 9.0 mL 10% KOH/ethanol (16.062 mmol KOH) in 60 mL ethanol was refluxed overnight under argon. It was then evaporated and the residue triturated with water. The solid crude product was filtered through a 250 mL silica pad using a 10% to 60% toluene-ethylacetate gradient to give 1.164 g (68%) of 6-bromo-2-(2,4-dimethylthiazol-5-yl)quinoline 8.3: ¹H-NMR (DMSO-d₆): δ (ppm) 8.39 (d, 1H, J=8.7 Hz), 8.27 (m, 1H), 7.88-7.86 (m, 3H), 2.68 (s, 3H), 2.64 (s, 3H). A DMSO solution of the product bromide, potassium acetate (3 eq.), (PPh₃)₂Pd(II)Cl₂ catalyst (0.05 eq.) and bis(neopentylglycolato)diboron (3 eq.) was heated at 50° C. under argon for 4 h. After 150 mL water and 150 mL ethyl acetate was added, the organic phase was separated. The aqueous phase was extracted one more time with 50 mL ethyl acetate. The organic phases were pooled and washed with water (2×), brine (2×) and dried (sodium sulfate). The solvent was evaporated and the residue was purified by filtering through a 400 mL silica pad using toluene-ethyl acetate gradient to get 4.4 g (84%) of the title compound 2.5

MS: 285.08 (M+H⁺);

¹H-NMR (DMSO-d₆): δ (ppm) 8.47 (d, 1H, J=8.7 Hz), 8.33 (s, 1H), 7.97 (m, 1H), 7.88-7.79 (m, 2H), 2.69 (s, 3H), 2.64 (s, 3H).

### Example 3

### Synthesis of 3-cyclohexyl-4-(2-(3-methoxy-6-(4′-chlorophenyl)phenyl)quinolin-6-yl)benzoic acid 1.8a

### Preparation of 3-cyclohexyl-4-methoxy-benzoic acid methyl ester 1.2

To 63 mL 0.5M ZnCl₂ solution in THF was added 15 mL 2M cyclohexyl-magnesium chloride at room temperature. The mixture was stirred for 20 minutes and then 22 mL NMP was added and the stirring was continued for 5 more minutes. 3-Iodo-4-methoxy-benzoic acid methyl ester 1.1 (2.92 g, 10 mmol) and 102 mg Pd(P(tBu)₃)₂ were added. The mixture was heated at 100° C. for 40 minutes. The solvent was evaporated and the residue was purified on silica gel using hexane-toluene gradient to yield 1.9 g (70%) 1.2. H¹-NMR (DMSO-d₆): δ(ppm) 7.78 (dd, 1H, J=8.4 and 2.4 Hz), 7.73 (d, 1H, J=2.4 Hz), 7.03 (d, 1H, J=8.7 Hz), 3.84 (s, 3H), 3.78 (s, 3H), 2.87 (m, 1H), 1.75 (m, 5H), 1.34 (m, 5H).

### Preparation of 3-cyclohexyl-4-hydroxy-benzoic acid methyl ester 1.3

To a solution of 1.7 g (6.85 mmol) 1.2 in 34 mL DCM was added 1M BBr₃ (34 mmol) at 0° C. then the mixture was stirred overnight at room temperature. 17 mL methanol was next added and the solution was evaporated to dryness. The residue was purified on a silica pad using toluene-EtOAc gradient. Yield: 1.57 g (92%) 1.3. MS: 233.13 (M−H⁺). H¹-NMR (DMSO-d₆): δ(ppm) 7.68 (d, 1H, J=2.1 Hz), 7.61 (dd, 1H, J=8.4 and 2.1 Hz), 6.84 (d, 1H, J=8.7 Hz), 3.75 (s, 3H), 2.83 (m, 1H), 1.76 (m, 5H), 1.32 (m, 5H).

### Preparation of 3-cyclohexyl-4-trifluoromethanesulfonyloxy-benzoic acid methyl ester 1.4

1.42 g (6.11 nmol) 1.3 in 30 mL DCM was treated at 0° C. with 1.98 mL pyridine, 74 mg DMAP, and 3 mL triflic anhydride. When the reaction reached completion the solvent was evaporated, the residue was dissolved in ethyl acetate, washed with water (1×) and brine (3×), dried with magnesium sulfate and evaporated to give 2.07 g (92%) 1.4 as a oil. H¹-NMR (DMSO-d₆): δ(ppm) 8.01 (d, 1H, J=2.1 Hz), 7.92 (dd, 1H, J=8.7 and 2.1 Hz), 7.52 (d, 1H, J=8.4 Hz), 3.86 (s, 3H), 2.77 (m, 1H), 1.83-1.29 (m, 10H). F¹⁹-NMR (DMSO-d₆): (ppm) 74.1.

### Preparation of 2-(4′-chloro-4-methoxy-biphenyl-2-yl)-quinoline-6-boronic acid 1.6

1.06 g (2.5 mmol) 1.5, 740 mg (7.5 mmol) potassium acetate, 87 mg (0.125 mmol) dichloro[1,1′-bis(triphenylphosphino)]palladium (II) dichloromethane adduct and 680 mg (3 mmol) bis(neopentyl glycolato)diboron were dissolved in 15 mL DMSO and the mixture was heated overnight at 95° C. for one day. The crude product was precipitated by addition of 30 mL water and purified on a silica gel pad using toluene-ethyl acetate solvent gradient elution to give 1.6 in quantitative yield. H¹-NMR (CDCl₃): δ (ppm) 8.27 (s, 1H), 8.16 (d, 1H, J=8.4 Hz), 7.98-7.89 (m, 2H), 7.40 (d, 1H, J=8.4 Hz), 7.25-7.02 (m, 7H), 3.85 (s, 3H).

### Preparation of 4-[2-(4′-chloro-4-methoxy-biphenyl-2-yl)-quinolin-6-yl]-3-cyclohexyl-benzoic acid methyl ester 1.7a

A mixture of 97 mg (0.25 mmol) 1.6, 92 mg (0.25 mmol) 1.4, 72 mg (0.0625 mmol) tetrakis(triphenylphosphino)-palladium(0), 5.5 mL methanol and 0.6 mL saturated sodium bicarbonate was heated under argon overnight at 80° C. The solvent was evaporated. The residue was triturated with water. The crude 1.7a was used in the following step without further purification. MS: 562.22 (M+H⁺).

### Preparation of 3-cyclohexyl-4-(2-(3-methoxy-5-(4′-chlorophenyl)phenyl)quinolin-6-yl)benzoic acid 1.8a

The whole amount of 1.7a from the previous step (0.25 mmol) was saponified by heating in a mixture of 2 mL 10% KOH/ethanol and 0.5 mL water at 80° C. for 30 minutes. The solvents were removed by evaporation and the residue was purified with RP-HPLC. Yield: 36 mg (27%). MS: 548.20 (M+H⁺). H¹-NMR (CDCl₃): di(ppm) 8.2 (d, 1H, J=8.7 Hz), 8.07 (d, 1H, J=8.7 Hz), 7.98 (d, 1H, J=1.5 Hz), 7.86 (d, 1H, J=1.5 Hz), 7.80 (dd, 1H, J=8.1 and 1.8 Hz), 7.70 (dd, 1H, J=8.7 and 2.1 Hz), 7.43 (d, 1H, J=8.7 Hz), 7.36 (d, 1H, J=7.8 Hz), 7.30-7.26 (m, 3H), 7.16-7.07 (m, 4H), 3.86 (s, 3H), 2.64 (m, 1H), 1.75-1.02 (m, 10H).

### Example 4

### Synthesis of 3-cyclohexyl-4-(2-(3-(2-methoxyethoxy)-6-(4′-chlorophenyl)phenyl)quinolin-6-yl)benzoic acid 1.8b

### Preparation of 4-[2-(4′-chloro-4-hydroxy-biphenyl-2-yl)-quinolin-6-yl]-3-cyclohexyl-benzoic acid methyl ester 1.7b

To a solution of 1.7a (0.4 g, 0.712 mmol) in anhydrous CH₂Cl₂ (30 mL) was added BBr₃ (0.471 mL, 4.98 mmol) under argon at −70° C. The mixture was stirred under Ar at −70° C. for 30 min and warmed to room temperature. The mixture was stirred at room temperature under argon overnight. MeOH (30 mL) was added dropwise at 0° C. After evaporation of solvent, the residue was dissolved in CHCl₃ (100 mL), which was washed with brine (30 mL 2×) and dried over Na₂SO₄. After evaporation of solvent, 1.7b was obtained (0.38 g, 98%). MS: 548.21 (M+H⁺). ¹H-NMR (CDCl₃): δ(ppm) 8.30 (d, 1H, J=8.4 Hz), 8.06 (d, 1H, J=1.5 Hz), 7.91 (d, 1H, J=9.0 Hz), 7.87 (dd, 1H, J=1.5, 7.8 Hz), 7.65-7.62 (m, 3H), 7.34 (d, 1H, J=8.1 Hz), 7.27 (d, 1H, J=8.1 Hz), 7.18 (d, 2H, J=8.7 Hz), 7.11 (d, 2H, J=8.7 Hz), 7.04-6.98 (m, 2H), 3.95 (s, 3H), 2.67 (m, 1H), 1.75-1.51 (m, 5H), 1.28-1.10 (m, 5H).

### Preparation of 3-cyclohexyl-4-(2-(3-(2-methoxyethoxy)-5-(4′-chlorophenyl)phenyl)quinolin-6-yl)benzoic acid 1.8b

A mixture of 1.7b (70 mg, 0.128 mmol), 2-bromoethyl methyl ether (60 μL, 0.638 mmol), and K₂CO₃ (88 mg, 0.638 mmol) in DMF (3 mL) was stirred at 80° C. for 1.5 h. After evaporation of solvent, the residue was dissolved in MeOH (2 mL) and 2 N NaOH (1 mL) was added. The reaction mixture was stirred at 60° C. for 2 h and cooled to 0° C. 5 N HCl was added to pH 3. After evaporation of solvent, the residue was purified by reverse phase HPLC to give 1.8b (40 mg, 53%). MS: 593.24, 592.24 (M+H⁺). ¹H-NMR (DMSO-d₆): δ (ppm) 8.27 (d, 1H, J=8.7 Hz), 8.11 (d, 1H, J=8.7 Hz), 7.99 (d, 1H, J=1.5 Hz), 7.91 (d, 1H, J=1.8 Hz), 7.82 (dd, 1H, J=1.8, 7.8 Hz), 7.74 (dd, 1H, J=1.8, 8.4 Hz), 7.45 (d, 1H, J=7.8 Hz), 7.37 (d, 1H, J=7.8 Hz), 7.34 (d, 1H, J=3.0 Hz), 7.29 (d, 2H, J=8.7 Hz), 7.20 (dd, 1H, J=3.0, 8.4 Hz), 7.16-7.11 (m, 3H), 4.42-4.21 (m, 2H), 3.72-3.69 (m, 2H), 3.34 (s, 3H), 2.64 (m, 1H), 1.76-1.69 (m, 4H), 1.63-1.43 (m, 3H), 1.28-1.03 (m, 3H).

### Example 5

### Synthesis of 3-cyclohexyl-4-(2-(2,4-dimethylthiazol-5-yl)quinolin-6-yl)-5-((E)-3-oxo-3-(piperidin-1-yl)prop-1-enyl)benzoic acid 2.9b

### Preparation of 3-cyclohexyl-4-hydroxy-5-iodo-benzoic acid methyl ester 2.1

To a solution of 1.3 (5 g, 0.214 mol) in AcOH (100 mL) was added NIS (5.55 g, 0.247 mol) in five portions. The reaction mixture was stirred at room temperature for 2.5 h and the solvent was evaporated to dryness. The product was purified by chromatography using CH₂Cl₂-hexanes (3:2) as eluent to give 2.1 (5.89 g, 76%). MS: 361.89 (M+H⁺).

### Preparation of 3-(2-tert-butoxycarbonyl-vinyl)-5-cyclohexyl-4-hydroxy-benzoic acid methyl ester 2.3

A mixture of 2.1 (0.7 g, 1.94 mmol), tert-butyl acrylate (1.14 mL, 9.7 mmol), (PPh₃)₂PdCl₂ (0.11 g, 0.155 mmol) and Et₃N (1.08 mL, 7.76 mmol) in DMF (25 mL) was stirred under argon at 80° C. for 5 h. The solvent was evaporated to dryness. The product was purified by chromatography using CH₂Cl₂-toluene (1:1) as eluent to give 2.3 (0.65 g, 93%). MS: 305.15 (M-55). ¹H-NMR (DMSO-d₆): δ (Ppm) 9.91 (s, 1H), 7.98 (s, 1H), 7.89 (d, 1H, J=15.60 Hz), 7.73 (s, 1H), 6.41 (d, 1H, J=15.90 Hz), 3.70 (s, 3H), 2.96 (m, 1H), 1.76-1.69 (m, 6H), 1.48-1.23 (m, 13H).

### Preparation of 3-(2-tert-Butoxycarbonyl-vinyl)-5-cyclohexyl-4-trifluoromethanesulfonyloxy-benzoic acid methyl ester 2.4

To a solution of 2.3 (0.6 g, 1.67 mmol) and Et₃N (0.52 mL, 3.75 mmol) in anhydrous CH₂Cl₂ (15 mL) was added Tf₂O dropwise under argon at −70° C. The reaction mixture was stirred under argon at −70° C. for 40 min and at room temperature for 1 h. The reaction was diluted with CH₂Cl₂ (150 mL) and washed with brine (50 mL 2×). The organic phase was dried over Na₂SO₄ and evaporated to give 2.4 (0.82 g, 100%).

### Preparation of 3-(2-carboxy-vinyl)-5-cyclohexyl-4-[2-(2,4-dimethyl-thiazol-5-yl)-quinolin-6-yl]-benzoic acid methyl ester, 2.7

Compound 2.4 was reacted with 2.5 to give 2.6. Compound 2.6 was then treated with trifluoroacetic acid according to the procedure described in example 10. Yield of 2.7 65%. MS: 527.23 (M+H⁺).

### Preparation of 2.8b

2.7 was reacted with piperidine to give 2.8b under conditions similar to those in example 10 (93%). MS: 594.31 (M+H⁺)

### Preparation of 3-cyclohexyl-4-(2-(2,4-dimethylthiazol-5-yl)quinolin-6-yl)-5-((E)-3-oxo-3-(piperidin-1-yl)prop-1-enyl)benzoic acid 2.9b

2.9b was prepared from 2.8b using the procedure described in example 10 (76%). MS: 579.5 (M+H⁺). ¹H-NMR (DMSO-d₆): δ (ppm) 8.50 (d, 1H, J=8.7 Hz), 8.29 (d, 1H, J=1.5 Hz), 8.07 (d, 1H, J=8.4 Hz), 7.97 (d, 1H, J=1.5 Hz), 7.93 (d, 1H, J=8.7 Hz), 7.83 (d, 1H, J=1.8 Hz), 7.57 (dd, 1H, J=1.8, 8.7 Hz), 7.12 (d, 1H, J=15.0 Hz), 6.98 (d, 1H, J=15.30 Hz), 3.52 (br s, 2H), 3.34 (br S, 2H), 2.74 (s, 3H), 2.71 (s, 3H), 2.26 (m, 1H), 1.70-1.34 (m, 14H), 0.92-0.82 (m, 2H).

### Example 6

### Synthesis of 3-cyclohexyl-4-(2-(2,4-dimethylthiazol-5-yl)quinolin-6-yl)-5-((E)-3-morpholino-3-oxoprop-1-enyl)benzoic acid 2.9a

### Preparation of 3-cyclohexyl-4-(2-(2,4-dimethylthiazol-5-yl)quinolin-6-yl)-5-((E)-3-morpholino-3-oxoprop-1-enyl)benzoic acid methyl ester 2.8a

Compound 2.7 was reacted with morpholine following the procedure of Example 5 to give 2.8a (89%). MS: 596.30 (M+H⁺).

### Preparation of 3-cyclohexyl-4-(2-(2,4-dimethylthiazol-5-yl)quinolin-6-yl)-5-((E)-3-morpholino-3-oxoprop-1-enyl)benzoic acid 2.9a.

2.9a was prepared from 2.8a under the similar reaction conditions as described in the Example 5 (85%). MS: 581.48 (M+H⁺). ¹H-NMR (DMSO-d₆): δ (ppm) 8.50 (d, 1H, J=8.4 Hz), 8.32 (d, 1H, J=1.2 Hz), 8.07 (d, 1H, J=8.7 Hz), 7.97 (d, 1H, J=1.2 Hz), 7.93 (d, 1H, J=8.7 Hz), 7.82 (d, 1H, J=1.8 Hz), 7.57 (dd, 1H, J=1.8, 8.7 Hz), 7.17 (d, 1H, J=15.0 Hz), 7.02 (d, 1H, J=15.30 Hz), 3.62 (br s, 2H), 3.51 (br s, 4H), 3.37 (br s, 2H), 2.74 (s, 3H), 2.70 (s, 3H), 2.27 (m, 1H), 1.70-1.18 (m, 8H), 0.93-0.82 (m, 2H).

### Example 7

### Synthesis of 3-cyclohexyl-5-ethynyl-4-(2-(2,4-dimethylthiazol-5-yl)quinolin-6-yl)benzoic acid methyl ester 4.4a

### Preparation of 3-cyclohexyl-4-hydroxy-5-trimethylsilanylethynyl-benzoic acid methyl ester 4.1

To a mixture of 2.1 (2.35 g, 6.52 mmol), CuI (0.25 mg, 1.3 mmol), (PPh₃)₄Pd (0.75 g, 0.652 mmol), and Et₃N (1.36 mL) in DMF under argon was added (trimethylsilyl)acetylene (5.57 mL, 0.0391 mol) dropwise over 1 h at room temperature. The reaction mixture was stirred under argon at room temperature overnight. After evaporation of solvent, the residue was purified by chromatography on silica gel using CH₂Cl₂-toluene (1:4) as eluent to give 4.1 (1.85 g, 86%). ¹H-NMR (CDCl₃): δ (ppm) 7.89 (d, 1H, J=2.1 Hz), 7.83 (d, 1H, J=2.1 Hz), 6.33 (s, 1H), 3.86 (s, 3H), 2.92 (m, 1H), 1.82-1.71 (m, 5H), 1.42-1.35 (m, 5H), 0.28 (s, 9H).

### Preparation of 3-cyclohexyl-4-[2-(2,4-dimethyl-thizol-5-yl)-quinolin-6-yl]-5-trimethylsilanyl-ethynyl-benzoic acid methyl ester 4.3

4.1 (1.64 g, 4.96 mmol) was reacted with Tf₂O (1.0 mL, 5.95 mmol) according to the procedure as described in the example 10 give 4.2 (2.29 g, 100%). 4.2 (2.29 g, 4.96 mmol) was reacted with 2.5 (1.61 g, 5.66 mmol) in the presence of (PPh₃)₂PdCl₂ (0.39 g, 0.34 mmol), K₃PO₄ (4.81 g, 22.64 mmol), and LiCl (0.264 mg, 6.23 mmol) in anhydrous 1,4-dioxane (40 mL) according the procedure as described in example 10 (2.1 g, 77%). ¹H-NMR (CDCl₃): δ (ppm) 8.36 (d, 1H, J=8.4 Hz), 8.28 (d, 1H, J=8.4 Hz), 8.24 (d, 1H, J=1.8 Hz), 8.19 (d, 1H, J=1.8 Hz), 7.88 (d, 1H, J=8.4 Hz), 7.85 (d, 1H, J=1.8 Hz), 7.72 (dd, 1H, J=1.5, 8.4 Hz), 4.13 (s, 3H), 2.97 (s, 3H), 2.92 (s, 3H), 2.66 (m, 1H), 1.91-1.61 (m, 5H), 1.43-1.20 (m, 5H), 0.18 (s, 9H).

### Preparation of 3-cyclohexyl-4-[2-(2,4-dimethyl-thiazol-5-yl)-quinolin-6-yl]-5-ethynyl-benzoic acid methyl ester 4.4a

To a solution of 4.3 (0.827 g, 1.496 mmol) in MeOH (20 mL) in an ice-bath was added a saturated aq. K₂CO₃ (0.5 mL). The mixture was stirred at room temperature for 1.5 h. Water (10 mL) was added and the mixture was neutralized to pH 7 with 5 M HCl. The solvent was evaporated to dryness. To the residue was added H₂O (20 mL). The precipitates were collected by filtration, washed with H₂O and dried to give 4.4a (0.68 g, 94%). MS: 481.33 (M+H⁺).

### Example 8

### Synthesis of of 3-cyclohexyl-5-ethynyl-4-(2-(2,4-dimethylthiazol-5-yl)quinolin-6-yl)benzoic acid 4.4b

4.3 (61 mg, 0.11 mmol) was treated 2N aq. NaOH in MeOH according to the procedure of example 10 to give 4.4b (28 mg, 55%). MS: 467.17 (M+H⁺). 1H-NMR (DMSO-d₆): δ (ppm) 8.51 (d, 1H, J=9.0 Hz), 8.03 (d, 1H, J=9.0 Hz), 7.97 (d, 1H, J=1.5 Hz), 7.91-7.87 (m, 3H), 7.61 (dd, 1H, J=1.8, 8.7 Hz), 3.95 (s, 1H), 2.72 (s, 3H), 2.68 (s, 3H), 2.35 (m, 1H), 1.71-0.87 (m, 10H).

### Example 9

### Synthesis of 3-cyclohexyl-4-(2-(2,4-dimethylthiazol-5-yl)-5-(1-morpholino-1-oxomethyl)-quinolin-6-yl)benzoic acid 4.6b

To a solution of 4.4a (0.4 g, 0.832 mmol) in CH₂Cl₂ (5 mL) was added tert-BuOH (6 mL), MeReO₃ (21 mg, 0.083 mmol) and 50% aq. H₂O₂ (0.163 mL, 2.83 mmol). The mixture was stirred at 45° C. for 16 h. After evaporation of solvent, the residue was dissolved in MeOH (20 mL) and hydrogenated over 10% Pd/C under 50 psi of H₂ for 5 h according to the procedure of the preparation of example 10. After separation by RP-HPLC to give 4.5a. MS: 501 (M+H⁺). 4.5a was coupled with morpholine and followed hydrolysis with 2N aq. NaOH according to example 10 to give 4.6b. MS: 556.24 (M+H⁺). ¹H-NMR (DMSO-d₆): δ(ppm) 8.45 (d, 1H, J=9.0 Hz), 8.02 (d, 1H, J=9.0 Hz), 7.98 (d, 1H, J=1.5 Hz), 7.90-7.85 (m, 1H), 7.79 (s, 1H), 7.63 (d, 1H, J=1.5 Hz), 7.58 (d, 1H, J=805 Hz), 3.54 (m, 4H), 2.75 (m, 4H), 2.72 (s, 3H), 2.66 (s, 3H), 2.42 (m, 1H), 1.67-0.92 (m, 10H).

### Example 10

### Synthesis of 3-cyclohexyl-4-(furan-3-yl)-5-((E)-3-oxo-3-(piperidin-1-yl)prop-1-enyl)benzoic acid 9.5a

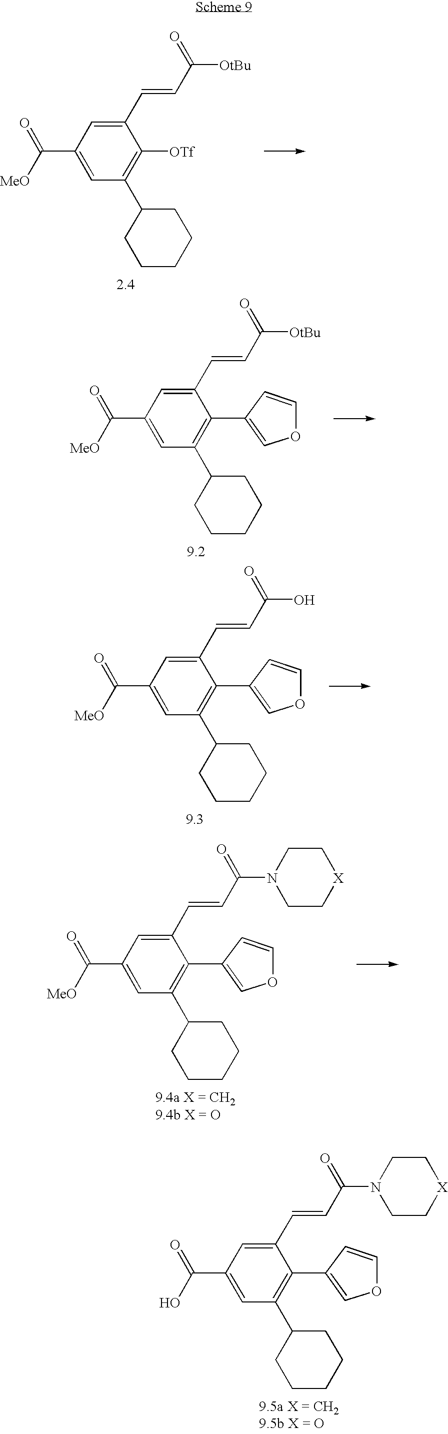

### Preparation of 3-(2-t-butoxy-vinyl)-5-cyclohexyl-4-furan-3-yl-benzoic acid methyl ester 9.2

A mixture of 9.1 (0.4 g, 0.812 mmol), furan-3-boronic acid (0.146 g, 1.3 mmol), (PPh₃)₄Pd (57 mg, 0.049 mmol), K₃PO₄ (0.69 g, 3.25 mmol), and LiCl (38 mg, 0.812 mmol) in anhydrous 1,4-dioxane (30 mL) was stirred under argon at 110° C. for 16 h. The solvent was evaporated to dryness. The product was purified by chromatography eluting with CH₂Cl₂ to give 9.2.

### Preparation of 3-(2-carboxy-vinyl)-5-cyclohexyl-4-furan-3-yl-benzoic acid methyl ester

To 9.2 was added anisole (0.2 mL) and TFA (3 mL). The mixture was stirred at room temperature for 1 h. After evaporation of solvent, the residue was purified by chromatography using CH₂Cl₂ to give a brown solid 9.3 (0.18 g, 96%). MS: 353.16 (M−H⁺).

### Preparation of 3-cyclohexyl-4-furan-3-yl-5-(3-oxo-3-piperidin-1-yl-propenyl)-benzoic acid methyl ester 9.4a

To a solution of 9.3 (0.1 g, 0.28 mmol) was added HBTU (0.14 g, 0.346 mmol) and diisopropylethylamine (0.123 mL, 0.7 mmol). The mixture was stirred at room temperature for 30 min. and piperidine (0.056 ml, 0.56 mmol) was added. The reaction mixture was stirred at room temperature for 1 h. After evaparation of solvent, the residue was passed a short silica gel pad eluting with CH₂Cl₂ to give 9.4a (92%). MS: 422.26 (M+H⁺).

### Preparation of 3-cyclohexyl-4-furan-3-yl-5-(3-oxo-3-piperidin-1-yl-propenyl)-benzoic acid 9.5a

9.4a (60 mg, 0.142 mmol) was dissolved in MeOH (1 mL)/THF (1 mL) and 2 N aq. NaOH was added. The mixture was stirred at 50° C. for 3 h and cooled to 0° C. 5 N HCl was added to pH 3. After evaporation of solvent, the residue was purified by reverse phase HPLC to give 9.5a (49 mg, 84%). MS: 407.25 (M+H⁺). ¹H-NMR (DMSO-d₆): δ(ppm) 8.19 (s, 1H), 7.88 (m, 2H), 7.68 (s, 1H), 7.27 (d, 1H, J=15.30 Hz), 7.11 (d, 1H, J=15.60 Hz), 6.50 (s, 1H), 3.58-3.45 (m, 4H), 2.50 (m, 1H), 1.73-1.08 (m, 16H).

### Example 11

### Synthesis of 3-cyclohexyl-4-furan-3-yl-5-(3-morpholin-4-yl-3-oxo-propenyl)-benzoic acid 9.5b

### Preparation of 3-cyclohexyl-4-furan-3-yl-5-(3-oxo-3-morpholino-propenyl)-benzoic acid methyl ester 9.4b

Compound 9.3 was reacted with morpholine under similar reaction conditions as described in the synthesis of 9.4a to give 9.4b (93%). MS: 424.23 (M+H⁺).

### Preparation of 3-cyclohexyl-4-furan-3-yl-5-(3-oxo-3-morpholino-propenyl)-benzoic acid 9.5b

Compound 9.5b was prepared as described in the synthesis of 9.5a (78%). MS: 409.22 (M+H⁺). ¹H-NMR (DMSO-d₆): δ (ppm) 8.22 (s, 1H), 7.88-7.87 (m, 2H), 7.68 (s, 1H), 7.32 (d, 1H, J=15.6 Hz), 7.13 (d, 1H, J=15.3 Hz), 6.50 (s, 1H), 3.57-3.48 (m, 8H), 2.53 (m, 1H), 1.74-1.64 (m, 4H), 1.45-1.09 (m, 6H).

### Example 12

### Synthesis of 3-Cyclohexyl-4-[2-(2,4-dimethyl-thiazol-5-yl)-quinolin-6-yl]-5-(2-morpholin-4-yl-2-oxo-ethyl)-benzoic acid (9)

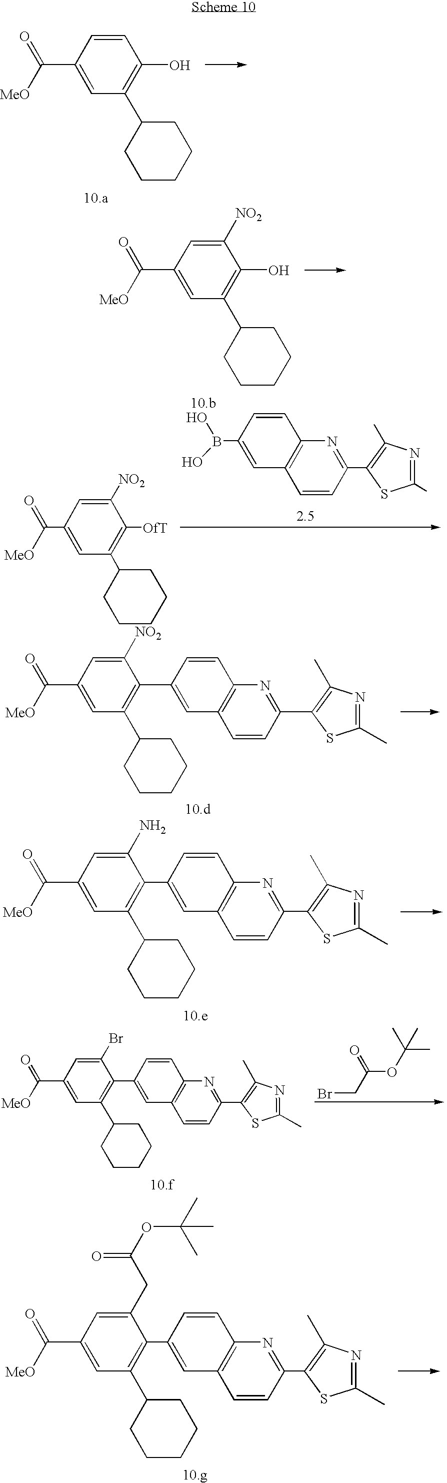

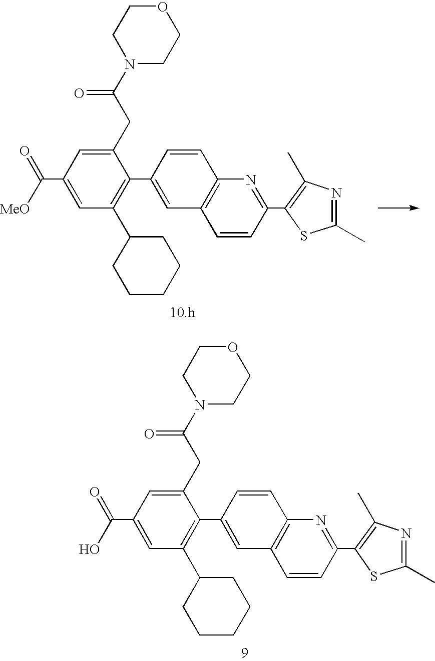

### Preparation of 3-cyclohexyl-4-hydroxy-5-nitro-benzoic acid methyl ester (10.b)

To a solution of 10.a (2.0 g, 8.53 mmol) in AcOH (50 mL) at 18° C. was added dropwise nitric acid. The mixture was stirred at room temperature for 1.5 h. After evaporation of the solvent, 10.b was obtained in a quantitative yield. MS: 280.1 (M+H⁺). ¹H-NMR (CDCl₃): δ (ppm) 11.34 (s, 1H), 8.66 (d, 1H, J=2.1 Hz), 8.12 (d, 1H, J=2.1 Hz), 3.93 (s, 3H), 3.06 (m, 1H), 1.91-1.77 (m, 6H), 1.50-1.26 (m, 4H).

### Preparation of 3-cyclohexyl-5-nitro-4-trifluoromethanesulfonyloxy-benzoic acid methyl ester (10.c)

To a solution of 10.b (1.14 g, 4.08 mmol) in anhydrous dichloromethane (15 mL) in the presence of triethylamine (2.84 mL, 20.4 mmol) was added trifluoromethanesulfonic anhydride (2.06 mL, 12.24 mmol) dropwise under Ar at −78° C. The mixture was then warmed to room temperature and stirred for 2 h. Dichloromethane (130 mL) was added and the solution was washed with H₂O (100 mL 2×), dried over anhydrous Na₂SO₄ and evaporated. A dark brown residue was separated by silica gel chromatography eluting with toluene-EtOAc (30:1) to give 10.c (0.84 g, 50% yield). MS: 412.1 (M+H⁺). ¹H-NMR (CDCl₃): δ (ppm) 8.47 (d, 1H, J=1.8 Hz), 8.29 (d, 1H, J=1.8 Hz), 3.94 (s, 3H), 3.15 (m, 1H), 1.92-1.89 (m, 5H), 1.51-1.44 (m, 5H).

### Preparation of 3-cyclohexyl-4-[2-(2,4-dimethyl-thiazol-5-yl)-quinolin-6-yl]-5-nitro-benzoic acid methyl ester (10.d)

A mixture of 10.c (1.0 g, 1.216 mmol), compound 2.5 (0.89 g, 3.16 mmol), Pd(PPh₃)₄ (tetrakis(triphenylphosphine)palladium)(1.68 mg, 0.0145 mmol), and saturated NaHCO₃ (1.5 mL) in MeOH (8 mL) was stirred under Ar at reflux for 16 h. After evaporation of the solvent, the residue was separated by silica gel chromatography eluting with hexanes-EtOAc (1:1) providing compound 10.d (0.92 g, 76%). MS: 502.2 (M+H⁺).

### Preparation of 3-amino-5-cyclohexyl-4-[2-(2,4-dimethyl-thiazol-5-yl)-quinolin-6-yl]-benzoic acid methyl ester (10.e)

Compound 10.d (1.0 g) was dissolved in EtOAc/MeOH(2:1, 40 mL) and hydrogenated under reduced pressure of 40 psi of H₂ over 5% Pd/C for 40 min. 10.e was obtained in a quantitative yield. MS: 472.2 (M+H⁺).

### Preparation of 3-bromo-5-cyclohexyl-4-[2-(2,4-dimethyl-thiazol-5-yl)-quinolin-6-yl]-benzoic acid methyl ester (10.f)

To a fine suspension of 10.e (0.81 g, 1.72 mmol) in acetone (35 mL) at 0° C. was added 48% aq. HBr (0.58 mL). A solution of NaNO₂ (0.148 g, 2.15 mmol) in water (1 mL) was added dropwise over 8 min. The mixture was stirred at 0° C. in an ice bath for another 10 min, and CuBr (30 mg) was added in three portions. The mixture was stirred at 5° C. for 15 min. After evaporation of the solvent, the residue was extracted with EtOAc (150 mL), washed with H₂O and dried over Na₂SO₄. The product was purified by chromatography (EtOAc-hexanes; 2:3) to give 10.f (0.69 g, 75%). MS: 537.1 (M+H⁺).

### Preparation of 3-tert-butoxycarbonylmethyl-5-cyclohexyl-4-[2-(2,4-dimethyl-thiazol-5-yl)-quinolin-6-yl]-benzoic acid methyl ester (10.g)

Zinc powder (0.5 g) was washed with 10% HCl, acetone and dried under a high vacuum overnight. Anhydrous THF (30 mL) was added. To the suspension in the presence of a catalytic amount of 12 was added tert-butyl bromoacetate (0.562 mL, 3.81 mmol) under Ar. The mixture was heated at 50° C. for 3 min and then stirred at room temperature for 2 h. This mixture was then transferred to a mixture of 10.f (0.68 g, 1.27 mmol), Pd(P(t-Bu)₃)₂ (33 mg) in NMP (5 mL) and anhydrous THF (60 mL) under Ar. The reaction mixture was stirred at 100° C. under Ar for 16 h. After filtration, the filtrate was evaporated to dryness and the residue was purified by chromatography (EtOAc-hexanes, 2:3) to give 10.g (0.69 g, 75%). MS: 537.1 (M+H⁺).

### Preparation of 3-cyclohexyl-4-[2-(2,4-dimethyl-thiazol-5-yl)-quinolin-6-yl]-5-(2-morpholin-4-yl-2-oxo-ethyl)-benzoic acid methyl ester (10.h)

To 10.g (0.654 g, 1.27 mmol) was added TFA (4 mL) and anisole (0.2 mL). The mixture was stirred at room temperature for 1 h. After evaporation of solvent, to the residue was added DMF (5 mL), HATU (0.72 g, 1.91 mmol) and diisopropylethylamine (0.44 mL, 2.54 mmol). The mixture was stirred at room temperature for 30 min and morpholine (0.22 mL, 2.54 mmol) was then added. The mixture was stirred at room temperature for 2 h. After evaporation of solvent, the product was purified by chromatography (from EtOAc-hexanes 1:1 to EtOAc). Yield 94%. MS: 584.3 (M+H⁺).

### Preparation of 3-cyclohexyl-4-[2-(2,4-dimethyl-thiazol-5-yl)-quinolin-6-yl]-5-(2-morpholin-4-yl-2-oxo-ethyl)-benzoic acid (7)

To a solution of 10.h (0.78 g, 1.34 mmol) in THF (8 mL) and MeOH (4 mL) was added 2N aq. NaOH (2.5 mL) and the mixture was stirred at 45° C. for 2 h. The mixture was neutralized with 5 N HCl to pH 6. After evaporation of solvent, the residue was washed with water (25 mL 2×) and dried to give compound 9 (30 mg, 39%). MS: 570.2 (M+H⁺). ¹H-NMR (DMSO-d₆): δ(ppm) 8.45 (d, 1H, J=8.7 Hz), 8.01 (d, 1H, J=8.4 Hz), 7.88 (d, 1H, J=8.7 Hz), 7.86 (d, 1H, J=1.2 Hz), 7.71-7.70 (m, 2H), 7.47 (dd, 1H, J=1.8, 8.7 Hz), 3.42 (d, 2H, J=4.5 Hz), 3.30-3.22 (m, 4H), 3.02-2.97 (m, 4H), 2.72 (s, 3H), 2.68 (s, 3H), 2.22 (m, 1H), 1.64-0.82 (m, 10H).

### Example 13

### Synthesis of 1-[3-cyclohexyl-4-[2-(2,4-dimethyl-thiazol-5-yl)-quinolin-6-yl]-5-(2-morpholin-4-yl-2-oxo-ethyl)-benzoylamino]-cyclopentanecarboxylic acid (10)

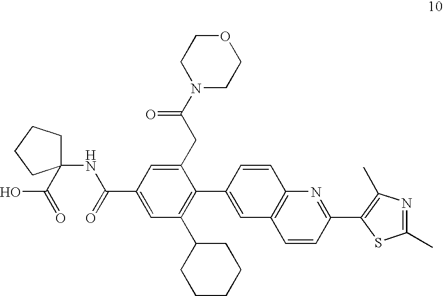

A mixture of compound 9 (50 mg, 0.0877 mmol), HATU (35 mg, 0.092 mmol) and diisopropylethylamine (38 μL, 0.22 mmol) in DMF (1.5 mL) was stirred at room temperature for 30 min and 1-amino-cyclopentane carboxylic acid (11.9 mg, 0.092 mmol) was then added. The mixture was stirred at room temperature for further 1 h. After evaporation of the solvent, the product was purified by HPLC (0-100%) to give compound 10 (8.4 mg, 14%). MS: 681.3 (M+H⁺). ¹H-NMR (DMSO-d₆): δ (ppm) 8.56 (s, 1H), 8.45 (d, 1H, J=8.7 Hz), 8.00 (d, 1H, J=8.4 Hz), 7.88 (d, 1H, J=8.7 Hz), 7.77 (d, 1H, J=1.5 Hz), 7.67 (d, 1H, J=1.8 Hz), 7.61 (d, 1H, J=1.5 Hz), 7.44 (dd, 1H, J=2.1, 8.7 Hz), 3.38 (d, 2H, J=6.6 Hz), 3.31-3.22 (m, 4H), 3.01-3.98 (m, 4H), 2.72 (s, 3H), 2.67 (s, 3H), 2.12-2.06 (m, 5H), 1.72-1.49 (m, 11H), 0.87 (m, 2H).

### Example 14

### Synthesis of 3-cyclohexyl-4-[2-(2,4-dimethyl-thiazol-5-yl)-quinolin-6-yl]-5-nitro-benzoic acid (7)

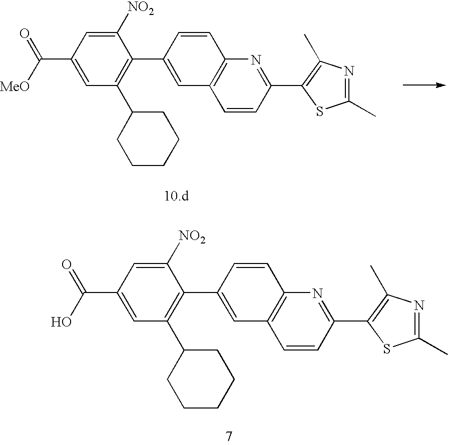

To a solution of 10.d (47 mg, 0.094 mmol) in THF (2 mL) and MeOH (1 mL) was added 2N aq. NaOH (0.5 mL) and the mixture was stirred at 65° C. for 2 h. The mixture was neutralized with 5 N HCl to pH 6. After evaporation of solvent, the residue was washed with water (25 mL 2×) and dried to give 7 (30.1 mg, 66%). MS: 488.1 (M+H⁺). ¹H-NMR (DMSO-d₆): δ (ppm) 8.46 (d, 1H, J=8.4 Hz), 8.27 (d, 1H, J=1.5 Hz), 8.23 (d, 1H, J=1.8 Hz), 8.04 (d, 1H, J=8.4 Hz), 7.89 (d, 1H, J=9.0 Hz), 7.87 (d, 1H, J=1.5 Hz), 7.75 (dd, 1H, J=2.1, 9.0 Hz), 2.72 (s, 3H), 2.67 (s, 3H), 2.36 (m, 1H), 1.78-1.42 (m, 7H), 1.23-1.19 (m, 1H), 0.97-0.87 (m, 2H).

### Example 15

### Synthesis of 3-amino-5-cyclohexyl-4-[2-(2,4-dimethyl-thiazol-5-yl)-quinolin-6-yl]-benzoic acid (8)

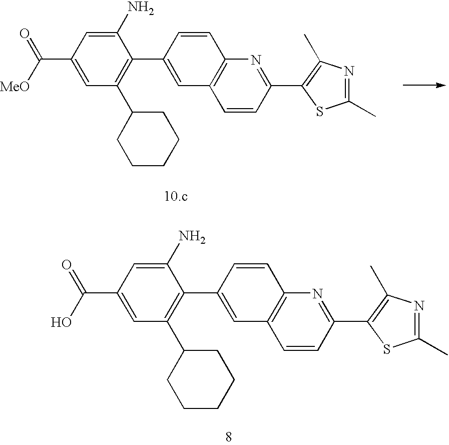

To a solution of 10.c (40 mg, 0.085 mmol) in THF (2 mL) and MeOH (1 mL) was added 2N aq. NaOH (0.5 mL) and the mixture was stirred at 65° C. for 2 h. The mixture was neutralized with 5 N HCl to pH 6. After evaporation of solvent, the residue was washed with water (25 mL 2×) and dried to give 8 (26.2 mg, 68%). MS: 458.2 (M+H⁺). ¹H-NMR (DMSO-d₆): δ(ppm) 8.46 (d, 1H, J=8.7 Hz), 8.04 (d, 1H, J=8.4 Hz), 7.86 (d, 1H, J=9.0 Hz), 7.79 (d, 1H, J=1.8 Hz), 7.51 (dd, 1H, J=1.5, 8.4 Hz), 7.24-7.21 (m, 2H), 2.71 (s, 3H), 2.66 (s, 3H), 2.16 (m, 1H), 1.62-0.82 (m, 10H).

### Example 16

### Synthesis of 2-[3-cyclohexyl-2-[2-(2,4-dimethyl-thiazol-5-yl)-quinolin-6-yl]-5-(1H-tetrazol-5-yl)-phenyl]-1-morpholin-4-yl-ethanone (11)

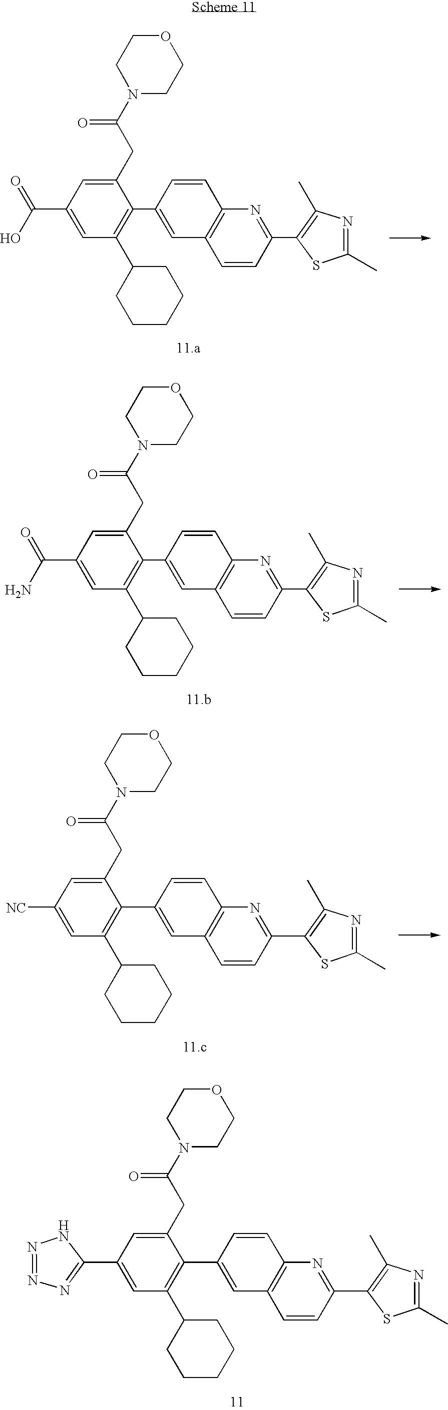

### Preparation of 3-Cyclohexyl-4-[2-(2,4-dimethyl-thiazol-5-yl)-quinolin-6-yl]-5-(2-morpholin-4-yl-2-oxo-ethyl)-benzamide (11.a)

A mixture of 11.a (0.142 g, 0.249 mmol), HBTU (0.189 g, 0.498 mmol) and diisopropylethylamine (0.14 mL, 0.8 mmol) in DMF (2 mL) was stirred at room temperature for 30 min and 2 M ammonia in 2-propanol (1.5 mL) was then added. The mixture was stirred at room temperature for further 1 h. After evaporation of the solvent, the residue was extracted with CHCl₃-MeOH (5:1, 100 mL 2×) and the organic phase was washed with water (50 ml×3), dried over anhydrous Na₂SO₄ and evaporated. The residue was purified by chromatography (CH₂Cl₂-MeOH, 6:1) to give 11.b(1.387 g, 98%). MS: 569.2 (M+H⁺).

### Preparation of 3-Cyclohexyl-4-[2-(2,4-dimethyl-thiazol-5-yl)-quinolin-6-yl]-5-(2-morpholin-4-yl-2-oxo-ethyl)-benzonitrile (11.c)

To a solution of 11.b (0.102 g, 0.179 mmol) in anhydrous DMF (1.5 mL) was added trifluoroacetic anhydride (75 μL, 0.537 mmol) and pyridine (38 μL, 0.716 mmol) at 0° C. The mixture was then stirred at room temperature for 6 h. After evaporation of solvent, the residue was separated by chromatogaraphy eluting with CH₂Cl₂-MeOH (10:1) to give 11.c. Yield 68%. MS: 551.2 (M+H⁺).

### Preparation of 2-[3-cyclohexyl-2-[2-(2,4-dimethyl-thiazol-5-yl)-quinolin-6-yl]-5-(1H-tetrazol-5-yl)-phenyl]-1-morpholin-4-yl-ethanone (11)

A mixture of 11.c (77 mg, 0.14 mmol) and azidotrimethyltin (43 mg, 0.207 mmol) in toluene (7 mL) was stirred under Ar at refluxing for 3 days. After evaporation of solvent, the residue was dissolved in MeOH (15 mL) and 5N HCl (1 mL) was added. The mixture was stirred at room temperature for 30 min and the solvent was evaporated. The residue was separated by chromatography eluting with CH₂Cl₂-MeOH (8:1) to give compound 11(38.1 mg, 46%). MS: 594.46 (M+H⁺). ¹H-NMR (DMSO-d₆): δ (ppm) 8.47 (d, 1H, J=8.4 Hz), 8.07 (br s, 1H), 8.03 (d, 1H, J=8.4 Hz), 7.90 (d, 1H, J=8.4 Hz), 7.84 (br s, 1H), 7.75 (br s, 1H), 7.51 (d, 1H, J=8.7 Hz), 3.45 (d, 2H, J=4.8 Hz), 3.31-3.23 (m, 4H), 3.03-2.99 (m, 4H), 2.73 (s, 3H), 2.70 (s, 3H), 2.25 (m, 1H), 1.74-1.52 (m, 6H), 1.22-1.16 (m, 2H), 0.94-0.85 (m, 2H).

### BIOLOGICAL EXAMPLES

### Example 1

### Anti-Hepatitis C Activity

Compounds can exhibit anti-hepatitis C activity by inhibiting HCV polymerase, by inhibiting other enzymes needed in the replication cycle, or by other pathways. A number of assays have been published to assess these activities. A general method that assesses the gross increase of HCV virus in culture is disclosed in U.S. Pat. No. 5,738,985 to Miles et al. In vitro assays have been reported in Ferrari et al. Jnl. of Vir., 73:1649-1654, 1999; Ishii et al., Hepatology, 29:1227-1235, 1999; Lohmann et al., Jnl of Bio. Chem., 274:10807-10815, 1999; and Yamashita et al., Jnl. of Bio. Chem., 273:15479-15486, 1998.

WO 97/12033, filed on Sep. 27, 1996, by Emory University, listing C. Hagedorn and A. Reinoldus as inventors, which claims priority to U.S. Provisional Patent Application Ser. No. 60/004,383, filed on September 1995, describes an HCV polymerase assay that can be used to evaluate the activity of the of the compounds described herein. Another HCV polymerase assay has been reported by Bartholomeusz, et al., Hepatitis C Virus (HCV) RNA polymerase assay using cloned HCV non-structural proteins; Antiviral Therapy 1996:1 (Supp 4) 18-24.

Screens that measure reductions in kinase activity from HCV drugs are disclosed in U.S. Pat. No. 6,030,785, to Katze et al., U.S. Pat. No. 6,228,576, Delvecchio, and U.S. Pat. No. 5,759,795 to Jubin et al. Screens that measure the protease inhibiting activity of proposed HCV drugs are disclosed in U.S. Pat. No. 5,861,267 to Su et al., U.S. Pat. No. 5,739,002 to De Francesco et al., and U.S. Pat. No. 5,597,691 to Houghton et al.

### Example 2

### Replicon Assay

A cell line, ET (Huh-lucubineo-ET) was used for screening of compounds of the present invention for HCV RNA dependent RNA polymerase. The ET cell line was stably transfected with RNA transcripts harboring a I389luc-ubi-neo/NS3-3′/ET; replicon with firefly luciferase-ubiquitin-neomycin phosphotransferase fusion protein and EMCV-IRES driven NS3-5B polyprotein containing the cell culture adaptive mutations (E1202G; T1280I; K1846T) (Krieger at al, 2001 and unpublished). The ET cells were grown in DMEM, supplemented with 10% fetal calf serum, 2 mM Glutamine, Penicillin (100 IU/mL)/Streptomycin (100 μg/mL), 1× nonessential amino acids, and 250 μg/mL G418 (“Geneticin”). They were all available through Life Technologies (Bethesda, Md.). The cells were plated at 0.5-1.0×104 cells/well in the 96 well plates and incubated for 24 hrs before adding nucleoside analogs. Then the compounds were added to the cells to achieve a final concentration of 5 or 50 μM. Luciferase activity was measured 48-72 hours later by adding a lysis buffer and the substrate (Catalog number Glo-lysis buffer E2661 and Bright-Glo leuciferase system E2620 Promega, Madison, Wis.). Cells should not be too confluent during the assay. Percent inhibition of replication was plotted relative to no compound control. Under the same condition, cytotoxicity of the compounds was determined using cell proliferation reagent, WST-1 (Roche, Germany). The compounds showing potent antiviral activities, but no significant cytotoxicities were chosen for further evaluation. For these determinations, a 10-point, 2-fold serial dilution for each compound was used which spans a concentration range of 1000 fold. IC50 and TC50 values were calculated by fitting % inhibition at each concentration to the following equation:
% inhibition=100%/[(IC50/[I])b+1]
where b is Hill's coefficient.

The % inhibition at a particular concentration was determined using the following equation:
% Inhibition=100−[100*(Lum with inhibitor-bg)/(Lum with no inhibitor-bg)]

where bg was the background with no replicon cell, and Lum was the luminescence intensity of the reporter luciferase gene.

In this assay, when tested at 12.5 μM, compounds 1.8b, 1.8a, 2.9b, 2.9a, 4.4b, 4.6b, 7, 8, 9, 10, and 11 exhibited 94%, 89%, 80%, 65%, 87%, 23%, 30%, 54%, 6.4%, 51% and 27% inhibitions, respectively.

### Example 3

### Cloning and Expression of Recombinant HCV-NS5b

The coding sequence of NS5b protein is cloned by PCR from pFKI389luc/NS3-3′/ET as described by Lohmann, V., et al. (1999) Science 285, 110-113 using the primers shown on page 266 of WO 2005/012288

The cloned fragment is missing the C terminus 21 amino acid residues. The cloned fragment is inserted into an IPTG-inducible expression plasmid that provides an epitope tag (His)₆ at the carboxy terminus of the protein.

The recombinant enzyme is expressed in XL-1 cells and after induction of expression, the protein is purified using affinity chromatography on a nickel-NTA column. Storage condition is 10 mM Tris-HCl pH 7.5, 50 mM NaCl, 0.1 mM EDTA, 1 mM DTT, 20% glycerol at −20° C.

### Example 4

### HCV-NS5b Enzyme Assay

The polymerase activity is assayed by measuring incorporation of radiolabeled UTP into a RNA product using a biotinylated, heteropolymeric template, which includes a portion of the HCV genome. Typically, the assay mixture (50 μL) contains 10 mM Tris-HCl (pH 7.5), 5 mM MgCl₂, 0.2 mM EDTA, 10 mM KCl, 1 unit/μL RNAsin, 1 mM DTT, 10 μM each of NTP, including [³H]-UTP, and 10 ng/μL heteropolymeric template. Test compounds are initially dissolved in 100% DMSO and further diluted in aqueous buffer containing 5% DMSO. Typically, compounds are tested at concentrations between 1 nM and 100 μM. Reactions are started with addition of enzyme and allowed to continue at 37° C. for 2 hours. Reactions are quenched with 8 μL of 100 mM EDTA and reaction mixtures (30 μL) are transferred to streptavidin-coated scintillation proximity microtiter plates (FlashPlates) and incubated at 4° C. overnight. Incorporation of radioactivity is determined by scintillation counting.

What is claimed is:

1. A compound of formula I, II, or III: 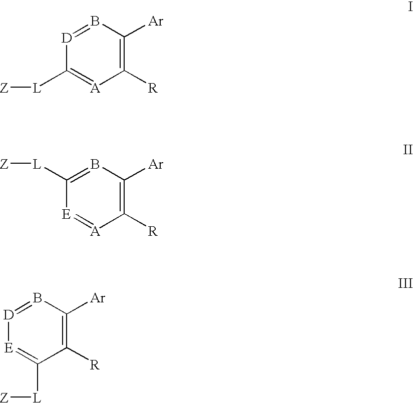
wherein:
B and D are independently N or C-L¹-R¹;
with the proviso that at least one of B or D is N or CH;
A and E are independently N or C—R²;
R is selected from the group consisting of alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl;
R¹ is selected from the group consisting of hydrogen, alkyl, substituted alkyl, cycloalkyl, cycloalkenyl, substituted cycloalkenyl, substituted cycloalkyl, alkenyl, substituted alkenyl, alkynyl, substituted alkynyl, heterocyclic, substituted heterocyclic, aryl, substituted aryl, heteroaryl, substituted heteroaryl, —COOH, —COOR^{1a}, —CONR³R⁴ and —NR³R⁴; where each of R^{1a}, R³, and R⁴ is independently selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl; or, alternatively, R³ and R⁴ may optionally be joined together with the nitrogen atom bound thereto to form a heterocyclic, substituted heterocyclic, heteroaryl or substituted heteroaryl group;
R² is selected from the group consisting of hydrogen, halo, C₁-C₂ alkyl, substituted C₁-C₂ alkyl, C₂-C₃ alkenyl, substituted C₂-C₃ alkenyl, cyclopropyl, and substituted cyclopropyl;
L and L¹ are independently selected from the group consisting of a bond, C₁-C₃ alkylene, substituted C₁-C₃ alkylene, C₂-C₃ alkenylene, substituted C₂-C₃ alkenylene, C₂-C₃ alkynylene, substituted C₂-C₃ alkynylene, C₃-C₆ cycloalkylene, substituted C₃-C₆ cycloalkylene, C₄-C₆ cycloalkenylene, C₄-C₆ substituted cycloalkenylene, arylene, substituted arylene, heteroarylene, and substituted heteroarylene;
Z is selected from the group consisting of:
(a) hydrogen, halo, alkyl, substituted alkyl, alkenyl, substituted alkenyl, alkoxy, substituted alkoxy, cyano, aryl, substituted aryl, heteroaryl, substituted heteroaryl, amino and substituted amino;
(b) COOH and COOR^{z}, wherein R^{z} is selected from the group consisting of alkyl, substituted alkyl, cycloalkyl, substituted cycloalkyl, alkenyl, substituted alkenyl, alkynyl, substituted alkynyl, heterocyclic, substituted heterocyclic, aryl, substituted aryl, heteroaryl, and substituted heteroaryl;
(c) —C(X¹)NR⁵R⁶, wherein X¹ is ═O, ═NH, or ═N-alkyl, R⁵ and R⁶ are independently selected from the group consisting of hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic or, alternatively, R⁵ and R⁶ together with the nitrogen atom pendent thereto, form a heterocyclic, a substituted heterocyclic, a heteroaryl or a substituted heteroaryl ring group;
(d) —C(X²)NR⁷S(O)₂R⁸, wherein X² is selected from ═O, ═NR⁹, and ═S, wherein R⁹ is hydrogen, alkyl, or substituted alkyl; R⁸ is selected from alkyl, substituted alkyl, aryl, substituted aryl, heteroaryl, substituted heteroaryl, heterocyclic, substituted heterocyclic, and NR¹⁰R¹¹ wherein each of R⁷, R¹⁰, and R¹¹ is independently hydrogen, alkyl, substituted alkyl, cycloalkyl, or substituted cycloalkyl, and wherein each of R⁷ and R¹⁰ is optionally substituted with one to three substituents selected from the group consisting of halo, hydroxyl, carboxy, carboxy ester, alkyl, alkoxy, amino, and substituted amino; or alternatively, R⁷ and R¹⁰ or R¹⁰ and R¹¹ together with the atoms bound thereto join together to form an optionally substituted heterocyclic group;
(e) —C(X³)—N(R¹²)CR¹³R¹³C(═O)R¹⁴, wherein X³ is selected from ═O, ═S, and ═NR¹⁵, where R¹⁵ is hydrogen or alkyl, R¹⁴ is selected from —OR¹⁶ and —NR¹⁰R¹¹ where R¹⁶ is selected from hydrogen, alkyl, substituted alkyl, alkenyl, substituted alkenyl, alkynyl, substituted alkynyl, aryl, substituted aryl, heteroaryl, substituted heteroaryl, heterocyclic and substituted heterocyclic; R¹⁰ and R¹¹ are as defined above;
R¹³ and R^{13′} are independently selected from hydrogen, alkyl, substituted alkyl, alkenyl, substituted alkenyl, alkynyl, substituted alkynyl, aryl, substituted aryl, cycloalkyl, substituted cycloalkyl, heteroaryl, substituted heteroaryl, heterocyclic, and substituted heterocyclic; or, alternatively, R¹³ and R^{13′} as defined are taken together with the carbon atom pendent thereto to form a cycloalkyl, substituted cycloalkyl, heterocyclic or substituted heterocyclic group; or, still further alternatively, one of R¹³ or R^{13′} is hydrogen, alkyl or substituted alkyl, and the other is joined, together with the carbon atom pendent thereto, with either the R¹⁶ and the oxygen atom pendent thereto or R¹⁰ and the nitrogen atom pendent thereto to form a heterocyclic or substituted heterocyclic group;
R¹² is selected from hydrogen and alkyl or, when R¹³ and R^{13′} are not taken together to form a ring and when R¹³ or R^{13′} and R¹⁰ or R¹¹ are not joined to form a heterocyclic or substituted heterocyclic group, then R¹², together with the nitrogen atom pendent thereto, may be taken together with one of R¹³ and R^{13′} to form a heterocyclic or substituted heterocyclic ring group;
(f) —C(X²)—N(R¹²)CR¹⁷R¹⁸R¹⁹, wherein X² and R¹² are defined above, and R¹⁷, R¹⁸ and R¹⁹ are independently alkyl, substituted alky, aryl, substituted aryl, heterocyclic, substituted heterocyclic, heteroaryl and substituted heteroaryl, or R¹⁷ and R¹⁸ together with the carbon atom pendent thereto form a cycloalkyl, substituted cycloalkyl, heterocyclic or substituted heterocyclic group; and
(g) carboxylic acid isostere;
with the proviso that when L is a bond, Z is not hydrogen; and
Ar is selected from the group consisting of aryl, heteroaryl, substituted aryl, and substituted heteroaryl;
or a pharmaceutically acceptable salt, ester, stereoisomer, prodrug, or tautomer thereof.

2. A compound of claim 1 wherein one of B and D is C-L′-R¹ and the other of B and D is CH.

3. A compound of claim 2 wherein A and E are C—R².

4. A compound of claim 3 wherein R² is hydrogen, L¹ is a bond, —CH₂—, —CH₂CH₂—, cis or trans —CH═CH—, cis or trans —(CH₃)C═CH—, cis or trans —CH═C(CH₃)—, or —CC—, and R¹ is hydrogen or —CONR³R⁴.

5. A compound of claim 4 wherein R³ and R⁴ together with the nitrogen atom bound thereto form a substituted or unsubstituted heterocyclic group.

6. A compound of claim 4 wherein R³ is alkyl and R⁴ is (heterocyclic)alkyl or (substituted heterocyclic)alkyl.

7. A compound of claim 4 wherein Z is —COOH, —COOR^{z}, 1H-tetrazol-5-yl, —C(O)NHSO₂CF₃,

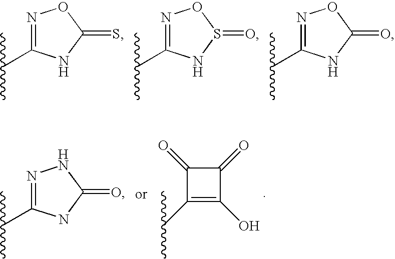

8. A compound of claim 7 wherein Z is —COOH.

9. A compound of claim 7 wherein L is a bond, —CH₂—, cis or trans —CH═CH—, cis or trans —(CH₃)C═CH—, or cis or trans —CH═C(CH₃)—.

10. A compound of claim 1 wherein R is substituted or unsubsituted cylcoalkyl, substituted or unsubsituted heterocyclic, or substituted and unsubstituted cyclohexyl.

11. A compound of claim 10 wherein R is cyclohexyl.

12. A compound of claim 1 wherein Ar is a substituted or unsubstituted five or six membered aryl or heteroaryl group, or a substituted or unsubstituted bicyclic [6,6], [5,6], or [6,5] aryl or heteroaryl group.

13. A compound of claim 1 having the formula V. 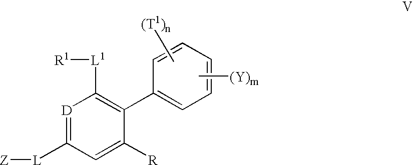
wherein D, Z, L, L¹, R, and R¹ are previously defined;
each T¹ is selected from the group consisting of alkyl, substituted alkyl, alkoxy, substituted alkoxy, amino, substituted amino, cyano, carboxy, carboxy ester, halo, hydroxyl, heterocyclic, substituted hetereocyclic, and nitro;
Y is selected from the group consisting of aryl, heteroaryl, substituted aryl, and substituted heteroaryl;
n is an integer equal to 0, 1, or 2; and
m is an integer equal to 0 or 1.

14. A compound of claim 13 having the formula 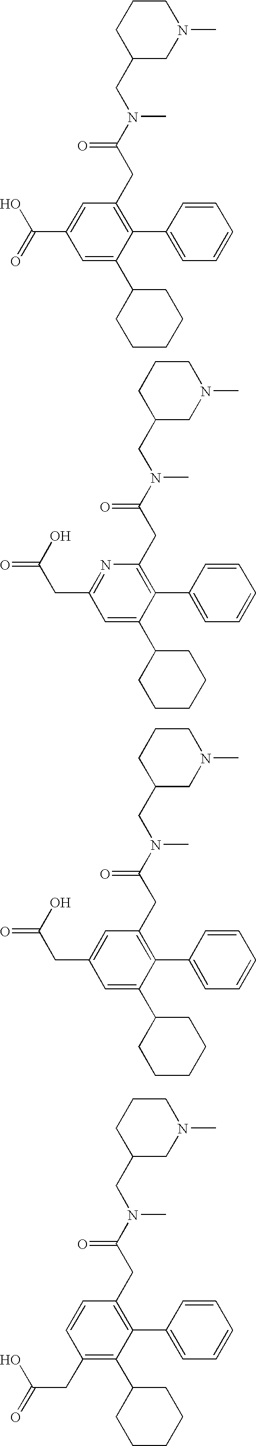 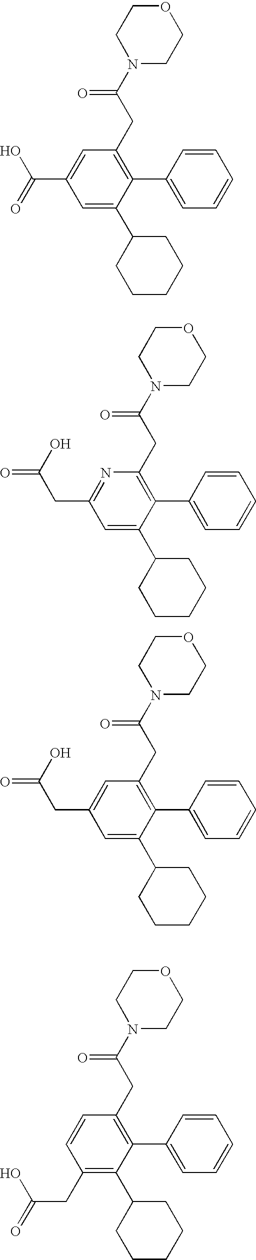 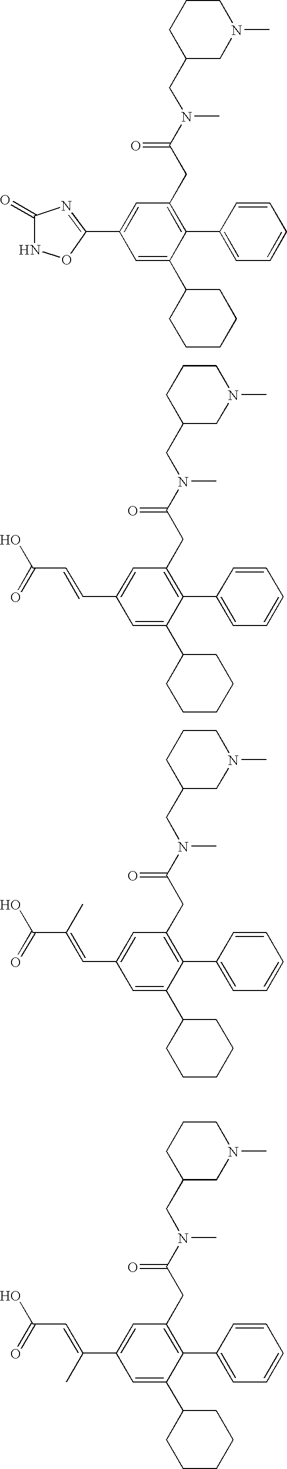 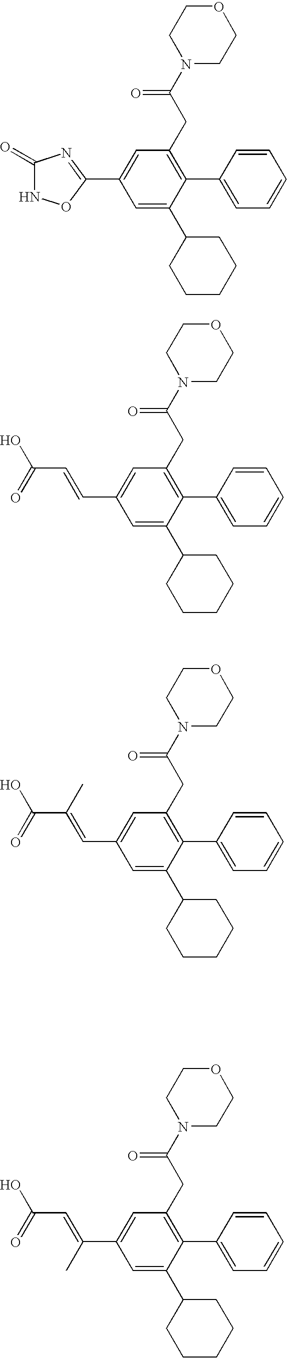
or the cis or trans stereiosomer thereof.

15. A compound of claim 1 wherein Ar is has the formula (H1) 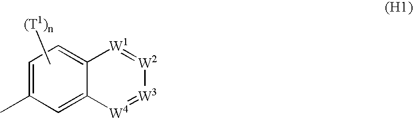
wherein each of W¹, W², W³ and W⁴ are independently selected from N,N-oxide, CH, CT², and C—Y; provided that no more than 2 of W¹, W², W³ and W⁴ are N; provided that one of W¹, W², W³ and W⁴ is C—Y; and further provided wherein no more than one N in (H1) is N-oxide;
each of T¹ and T² are independently selected from the group consisting of alkyl, substituted alkyl, alkoxy, substituted alkoxy, amino, substituted amino, cyano, carboxy, carboxy ester, halo, hydroxyl, heterocyclic, substituted hetereocyclic, and nitro;
Y is selected from the group consisting of aryl, heteroaryl, substituted aryl, and substituted heteroaryl; and
n is an integer equal to 0, 1, or 2.

16. A compound of claim 15 wherein Ar has the formula (H2) 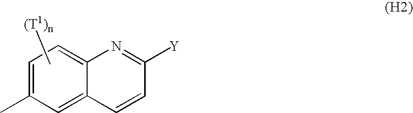
wherein T¹, n, and Y are previously defined.

17. A compound of claim 1 having the formula VI: 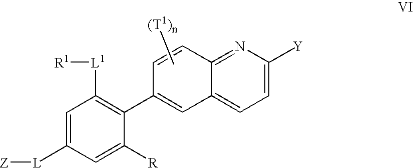
wherein Z, L, L¹, R, and R¹ are previously defined;
each T¹ is selected from the group consisting of alkyl, substituted alkyl, alkoxy, substituted alkoxy, amino, substituted amino, cyano, carboxy, carboxy ester, halo, hydroxyl, heterocyclic, substituted hetereocyclic, and nitro;
Y is selected from the group consisting of aryl, heteroaryl, substituted aryl, and substituted heteroaryl; and
n is an integer equal to 0, 1, or 2.

18. A compound of claim 17 wherein R is substituted or unsubsituted cylcoalkyl, substituted or unsubsituted heterocyclic, or substituted and unsubstituted cyclohexyl.

19. A compound of claim 18 wherein R is cyclohexyl.

20. A compound of claim 17 wherein Z is COOH and L is a bond, —CH₂—, cis or trans —CH═CH—, cis or trans —(CH₃)C═CH—, or cis or trans —CH═C(CH₃)—.

21. A compound of claim 17 wherein R¹ is H or —CONR³R⁴ and L¹ is a bond, cis or trans —CH═CH—, or cis or trans —(CH₃)C═CH—.

22. A compound of claim 21 wherein R¹-L¹ is H, (E)-3-oxo-3-(piperidin-1-yl)prop-1-enyl, (E)-3-morpholino-3-oxoprop-1-enyl, or ethynyl.

23. A compound of claim 17 wherein Y is substituted aryl or substituted heteroaryl.

24. A compound of claim 23 wherein Y is selected from the group consisting of 3-(2-methoxyethoxy)-5-(4′-chlorophenyl)phenyl, 3-methoxy-5-(4′-chlorophenyl)phenyl, and 2,4-dimethylthiazol-5-yl.

25. The compound of claim 17 selected from the group consisting of:
3-cyclohexyl-4-(2-(3-(2-methoxyethoxy)-6-(4′-chlorophenyl)phenyl)quinolin-6-yl)benzoic acid;
3-cyclohexyl-4-(2-(3-methoxy-6-(4′-chlorophenyl)phenyl)quinolin-6-yl)benzoic acid;
3-cyclohexyl-4-(2-(2,4-dimethylthiazol-5-yl)quinolin-6-yl)-5-((E)-3-oxo-3-(piperidin-1-yl)prop-1-enyl)benzoic acid;
3-cyclohexyl-4-(2-(2,4-dimethylthiazol-5-yl)quinolin-6-yl)-5-((E)-3-morpholino-3-oxoprop-1-enyl)benzoic acid;
3-cyclohexyl-α-ethynyl-4-(2-(2,4-dimethylthiazol-6-yl)quinolin-6-yl)benzoic acid;
3-cyclohexyl-4-(2-(2,4-dimethylthiazol-5-yl)-5-(1-morpholino-1-oxomethyl)-quinolin-6-yl)benzoic acid;
3-cyclohexyl-4-[2-(2,4-dimethyl-thiazol-5-yl)-quinolin-6-yl]-5-nitro-benzoic acid;
3-amino-5-cyclohexyl-4-[2-(2,4-dimethyl-thiazol-5-yl)-quinolin-6-yl]-benzoic acid;
3-cyclohexyl-4-[2-(2,4-dimethyl-thiazol-yl)-quinolin-6-yl]-5-(2-morpholin-4-yl-2-oxo-ethyl)-benzoic acid;
1-[3-cyclohexyl-4-[2-(2,4-dimethyl-thiazol-5-yl)-quinolin-6-yl]-5-(2-morpholin-4-yl-2-oxo-ethyl)-benzoylamino]-cyclopentanecarboxylic acid;
2-[3-cyclohexyl-2-[2-(2,4-dimethyl-thiazol-5-yl)-quinolin-6-yl]-5-(1H-tetrazol-5-yl)-phenyl]-1-morpholin-4-yl-ethanone;
or the cis or trans stereiosomer thereof.

26. The compound of claim 17 selected from the group consisting of:
3-(4-{2-[4′-chloro-4-(2-methoxy-ethoxy)-biphenyl-2-yl]-quinolin-6-yl}-3-cyclohexyl-phenyl)-acrylic acid;
3-{4-[2-(4′-chloro-4-methoxy-biphenyl-2-yl)-quinolin-6-yl]-3-cyclohexyl-phenyl}-acrylic acid;
3-[3-cyclohexyl-4-[2-(2,4-dimethyl-thiazol-5-yl)-quinolin-6-yl]-5-(3-oxo-3-piperidin-1-yl-propenyl)-phenyl]-acrylic acid;
3-[3-cyclohexyl-4-[2-(2,4-dimethyl-thiazol-5-yl)-quinolin-6-yl]-5-(3-morpholin-4-yl-3-oxo-propenyl)-phenyl]-acrylic acid;
3-{3-cyclohexyl-4-[2-(2,4-dimethyl-thiazol-5-yl)-quinolin-6-yl]-5-ethynyl-phenyl}-acrylic acid;
3-[3-cyclohexyl-4-[2-(2,4-dimethyl-thiazol-5-yl)-quinolin-6-yl]-5-(morpholine-4-carbonyl)-phenyl]-acrylic acid;
3-(4-{2-[4′-chloro-4-(2-methoxy-ethoxy)-biphenyl-2-yl]-quinolin-6-yl}-3-cyclohexyl-phenyl)-but-2-enoic acid;
3-{4-[2-(4′-chloro-4-methoxy-biphenyl-2-yl)-quinolin-6-yl]-3-cyclohexyl-phenyl}-but-2-enoic acid;
3-[3-cyclohexyl-4-[2-(2,4-dimethyl-thiazol-5-yl)-quinolin-6-yl]-5-(3-oxo-3-piperidin-1-yl-propenyl)-phenyl]-but-2-enoic acid;
3-[3-cyclohexyl-4-[2-(2,4-dimethyl-thiazol-5-yl)-quinolin-6-yl]-5-(3-morpholin-4-yl-3-oxo-propenyl)-phenyl]-but-2-enoic acid;
3-{3-cyclohexyl-4-[2-(2,4-dimethyl-thiazol-5-yl)-quinolin-6-yl]-5-ethynyl-phenyl}-but-2-enoic acid;
3-[3-cyclohexyl-4-[2-(2,4-dimethyl-thiazol-5-yl)-quinolin-6-yl]-5-(morpholine-4-carbonyl)-phenyl]-but-2-enoic acid;
or the cis or trans stereiosomer thereof.

27. A pharmaceutical composition comprising a pharmaceutically acceptable carrier and a therapeutically effective amount of a compound of claim 1 or a mixture of two or more of such compounds.

28. A method for treating or preventing a viral infection in a mammal mediated at least in part by a virus in the Flaviviridae family of viruses which method comprises administering to a mammal a pharmaceutical composition according to claim 27.

29. The method of claim 28 wherein said viral infection is a hepatitis C viral infection.

30. The method of claim 28 in combination with the administration of a therapeutically effective amount of one or more agents active against hepatitis C virus.

31. The method of claim 30 wherein said active agent against hepatitis C virus is an inhibitor of HCV proteases, HCV polymerase, HCV helicase, HCV NS4B protein, HCV entry, HCV assembly, HCV egress, HCV NS5A protein, or inosine 5′-monophosphate dehydrogenase.

32. The method of claim 31 wherein said agent active against hepatitis C virus is interferon-alpha or pegylated interferon-alpha alone or in combination with ribavirin or levovirin.
